(19) United States
(12) Patent Application Publication
(10) Pub. No.: US 2023/0140994 A1
(43) Pub. Date: May 11, 2023

(54) REPLICATION-DEFICIENT AVIAN ADENOVIRAL VECTORS, THEIR DESIGN AND USES

(71) Applicant: Greffex, Inc., Houston, TX (US)

(72) Inventor: Uwe D. STAERZ, Denver, CO (US)

(21) Appl. No.: 17/907,574

(22) PCT Filed: Mar. 29, 2021
(86) PCT No.: PCT/US2021/024576

§ 371 Date: Sep. 28, 2022

Related U.S. Application Data
(60) Provisional application No. 63/001,361, filed on Mar. 29, 2020.

(51) Int. Cl. C12N 15/861

(52) U.S. Cl. CPC C12N 15/861; C12N 2710/10221; C12N 2710/10252; C12N 2710/10243

(57) ABSTRACT

The embodiments disclosed herein relate to the design, engineering and production of replication-deficient gene delivery vectors that are based on aviadenoviruses. More particularly their use is described in the transfer of genes, genetic engineering of cells and animals, the expression of proteins the development of vaccines. In some embodiment, the designs and packaging of partially deleted aviadenovirus vectors are disclosed. In other embodiments, the designs and packaging of fully deleted aviadenovirus vectors, the propagation of replication-deficient aviadenovirus vectors, and the characteristic and engineering of host cells are disclosed. In other embodiments, the use of such vectors in veterinary medicine is described.

### FIELD

The present application relates to the composition of gene transfer vectors and their uses for the transfer of nucleic acids into cells, tissues and organs, in particular for applications in veterinary medicine, such as genetic engineering and vaccination of animals.

### BACKGROUND

Animal and human adenoviruses have been widely studied as infectious agents, as subjects of basic research, and for their potential use in gene medicine, such as gene therapy and vaccination. There are five genera of adenoviridae. They are the animal ones, the Atadenovirus, Aviadenovirus (AAd, birds), Ichtadenovirus, Siadenovirus and Mastadenovirus. The best examined examples are the mastadenoviruses, i.e. the human adenoviruses, for which forty-nine serotypes have been identified and categorized into six species or subgenera (A through F). The aviadenoviruses (AAd) or avian adenoviruses primarily affect animals of the class aves or birds. Currently eight species and ten serotypes of AAd have been identified.

Adenoviral genomes are flanked on both sides by inverted terminal repeat sequences (LITR and RITR), which are essential to replication of adenoviruses. A packaging signal called Ψ is located adjacent to the LITR. The infectious cycle of adenoviruses is divided in an early and a late phase, as exemplified for a standard adenovirus, such as the human adenovirus of the serotype 5. In an early phase, the virus is uncoated and genome transported to the nucleus, after which the early gene regions (E), E1, E2, E3 and E4 or their equivalents become transcriptionally active. E1 contains two regions named E1A and E1B. The E1A region (sometimes referred to as immediate early region) encodes two major proteins that are involved in modification of the host-cell cycle and activation of the other viral transcription regions. The E1 B region encodes two major proteins, 19K and 55K, that prevent, via different routes, the induction of apoptosis resulting from the activity of the E1A proteins. In addition, the E1B-55K protein is required in the late phase for selective viral mRNA transport and inhibition of host protein expression. E2 is also divided in E2A and E2B region that together encode three proteins. DNA binding protein, viral polymerase and pre-terminal protein, are all involved in the replication of the viral genome. The E3 region is not required for replication in vitro, but encodes several proteins that subvert the host defense mechanism toward viral infection. The E4 region encodes at least six proteins involved in several distinct functions related to viral mRNA splicing and transport, host cell mRNA transport, viral and cellular transcription and transformation.

The late proteins necessary for formation of the viral capsids and packaging of the viral genome, are all generated from the major late transcription unit that becomes fully active after the onset of viral DNA replication. A complex process of differentiated splicing and polyadenylation gives rise to more than 15 mRNA species that share a tripartite Ieader sequence. The early proteins E1B-55K and E4-Orf3 and Orf6 play a pivotal role in the regulation of late mRNA processing and transport from the nucleus. Packaging of newly formed adenoviral genomes in pre-formed capsids is mediated by at least two adenoviral proteins, the late 52/55k and an intermediate protein 1Va2, through interaction with the viral packaging signal Ψ located at the left end of the Ad5 genome. A second intermediate protein pIX is part of the capsid and is known to stabilize the hexon-hexon interactions. In addition, pIX has been described to transactivate TATA-containing promoters like the EIA promoter and the major late promoter (MLP).

One of the most well defined AAd is the chicken embryo lethal orphan (CELO) virus, which represents serotype 1 of AAd. The CELO virus genome is 43,804 bp long and has been completely sequenced, and its transcriptional organization has been established (FIG. 1). The central region of the viral genome is strongly homologous with other adenoviruses: the lower strand encodes replication functions (DNA polymerase, DNA-binding protein, pTP), and the upper strand, which is transcribed under the control of a single major late promoter (MLP), encodes capsid structural proteins. On either side of this central part there are two regions encoding at least 22 open reading frames (ORFs) that have no sequence homology with the E1, E3, and E4 regions of mammalian adenoviruses. Only 2 of these 22 genes have been studied: ORF8 encodes the GAM-1 protein, which was identified as a functional homolog to human adenovirus E1B 19K protein, and ORF22 encodes a protein that interacts with the retinoblastoma protein, which is similar to human adenovirus E1A protein, and cooperates with GAM-1 to activate the E2F pathway.

With the information about the function of some of the ORFs of CELO, it was established which CELO genome segments and ORFs were essential for viral replication of the CELO virus. It was also investigated whether the conservation of such genome segments provided the replication-competency of CELO-based vectors and therefore allowed the construction of a CELO-based replication-competent AAd gene transfer vector. It was shown that an expression cassette for foreign genes could be inserted into this region to generate a replication-competent gene delivery vector suited for vaccine applications in birds. It was also found that a CELO based replication-competent vector infected human cells and thus may also infect human subjects with yet unknown consequences. In other studies segments of the CELO genome were identified that were required for the replication of the genome. It was also shown that the deleted fragments ciykd could be provided in trans to drive replication and packaging of the partially deleted replication-deficient CELO genome.

Adenovirus-Based Vectors and Adenoviral Packaging Cell Lines

Adenovirus-based vectors have been used as a means to achieve high level gene transfer into various cell types, as vaccine delivery vehicles, for gene transfer into tissue transplants, for gene therapy, and to express recombinant proteins in cell lines and tissues that are otherwise difficult to transfect with high efficiency. Current systems for packaging replication-deficient human adenovirus-based vectors deleted of E1, consist of a host cell and a source of the adenoviral late genes. The current known human host cell lines, including the HEK293, OBI, and PERC.6 cells, express only early (nonstructural) adenovirus genes, not the late adenoviral (structural) genes needed for packaging. The adenoviral late genes are provided either by the adenoviral vectors themselves in cis or by a helper adenoviral virus in trans. The adenoviral vectors that provide the genes themself necessary for their encapsidation carry minimally modified adenoviral genomes principally deleted of the E 1 and some cases also the E3 and other adenoviral regions. In the case of replication-competent adenoviral vectors non-modified host cells have been used, such as the human A549 or the chicken hepatocarcinoma cell (LMH). In the case of replication-deficient adenoviral vectors the host cells were provide with gene expression constructs that delivered segments of the left end of the adenoviral genome, such as but not limited to, the E1 genes.

More recently, fully deleted “gutless” adenoviral vectors that are devoid of all viral protein-coding DNA sequences have been developed. The “gutless” adenoviral vectors contain only the ends of the viral genome (LITR and RITR), genes of interest, such as therapeutic genes, and the normal packaging recognition signal (Ψ), which allows this genome to be selectively packaged. However, to propagate the “gutless” adenoviral vector required a helper adenovirus that contains the adenoviral genes required for replication and virion assembly as well as LITR, RITR, and Ψ While this helper virus-dependent system allows the introduction of large heterologous genetic material, in the case of a fully deleted human adenoviral vector of up to about 35 kb, the helper virus contaminates the preparations of “gutless” adenoviral vectors using this approach. Contaminating replication competent helper viruses pose serious problems for gene therapy, vaccine, and transplant applications both because of the replication competent virus and because of the host's immune response to the adenoviral genes in the helper virus. One approach to decrease helper contamination in this helper virus-dependent vector system has been to introduce a conditional gene defect in the packaging recognition signal (Ψ) making it less likely that its DNA is packaged into a virion.

Fully deleted “gutless” Adenoviral vectors produced in such systems still have significant contamination with helper virus. A novel technology replaces the helper viruses with a packaging expression plasmid that is the deleted of the packaging signal T. Co-transfection of the vector genome together with the packaging expression plasmid into host cells is used to initiate vector encapsidation. This system apriori prevents contamination with the helper virus and at the same time limits the viral recombination that often results in the appearance of replication-competent viruses. Being able to produce “gutless” adenoviral gene transfer vectors without helper virus contamination eliminated helper virus contamination results in reduced vector toxicity and prolonged gene expression in human subjects and animals.

It is believed that adenoviral genes especially adenoviral late genes carried in minimally modified adenoviral vectors or in adenoviral helper viruses: 1) contribute to the inflammatory response seen after adenoviral mediated gene therapy, 2) decrease the immune response towards the gene of interest in vaccine applications, 3) interfere with normal cellular functions, and 4) result in protein contaminants in protein expression applications. Further, endogenous adenoviral genes occupy space in minimally modified adenoviral vectors that could be beneficially be used for carrying other genetic information. Remarkable progress has been made with adenoviral vectors in the last decade, but serious shortcomings continue to challenge their use.

Adenovirus Vectors for Gene Therapy and Protein Expression

Gene delivery or gene therapy is a promising method for the treatment of acquired and inherited diseases. An ever-expanding array of genes for which abnormal expression is associated with life-threatening human diseases are being cloned and identified. The ability to express such cloned genes will ultimately permit the prevention and/or cure of many important human diseases, diseases for which current therapies are either inadequate or nonexistent.

Following an initial administration of adenoviral vector, serotype-specific antibodies are generated against epitopes of the major viral capsid proteins, namely the penton, hexon and fiber. Given that such capsid proteins are the means by which the adenovirus attaches itself to a cell and subsequently infects the cell, such antibodies are then able to block or “neutralize” reinfection of a cell by the same serotype of adenovirus or adenoviral vector. This may necessitate using a different serotype of adenovirus in order to administer one or more subsequent doses of exogenous therapeutic DNA in the context of gene therapy and vaccines. In addition, both therapeutic and viral gene products are expressed on target cells when using minimally modified adenoviral vectors or adenoviral helper virus contaminated adenoviral vector preparations. These antigens can be recognized by cellular immune responses leading to the destruction of the transduced cells or tissues and thus the beneficial effect of gene therapy and vaccination may be negated. As a result of these immune-related obstacles the widespread use of minimally modified viral vectors has been stymied.

At least 53 different forms of human adenovirus and in addition numerous animal adenoviruses have been characterized. The principal discriminating factor among these viruses is the humoral immune (i.e. antibody) response to the capsid hexon protein (encoded by various alleles of the L3 gene). In fact, the majority of variation among the different hexon proteins occurs in three hyper-variable regions; the humoral immune response to Adenoviruses is centered on these hypervariable regions. Other structures, such as the fiber proteins on the adenoviral surface can also be recognized by the humoral immune systems. The interference of humoral immune responses with the activity of minimally modified adenoviral vectors can therefore be mitigate by switching adenoviral serotypes between each application. Late adenoviral genes show less variability and therefore T cell responses induced by minimally modified adenoviral vectors or adenoviral helper viruses cannot be avoided by switching the adenoviral serotype of the vectors.

Human populations have been exposed to natural adenovirus infections of certain adenoviral serotypes. Therefore, these subjects carry humoral and cellular immune responses directed genes expressed by these adenoviruses and adenoviral vectors based on adenoviruses of these serotypes. Two advances have sought to overcome the problems. They are the use of “gutless” (fully deleted) adenoviral vectors and the use adenoviral vectors based on rare or animal adenoviruses expressing rare or animal serotypes. While the use of “gutless” adenoviral vectors removes the adenoviral genes, such as L3, from the therapeutic vector, the propagation of these “gutless” adenoviral vectors requires the presence of helper adenoviruses that still carry the adenoviral genes. These helper viruses are significant contaminants in the preparations of “gutless” adenoviral vectors. The use of minimally modified adenoviral vectors based on rare or animals serotypes may avoid the problem of pre-existing humoral immunity and possibly to a lesser extent pre-existing cellular immunity in that subjects who have been previously been exposed to an adenovirus of a given serotype. Still, as the minimally modified adenoviral vectors express adenoviral genes including the highly immunogenic L3, they may induce potent humoral and cellular immune responses to these adenoviral genes. Therefore, repeated applications of a minimally modified adenoviral vector of a given serotype will not be possible.

Therefore, adenoviral vectors of animal origin have been investigated. They may be useful when treating humans as they may not have been exposed to the animal virus serotype. In addition, animal adenoviral vectors may be better suited to be used in the respective animal as the adenovirus has been selected to efficiently infect this animal. For instance, aviadenovirus-based vectors may be more efficient as vaccine carriers for birds than adenovirus-vectors based on human adenoviruses. Furthermore, aviadenovirus-based vectors may have an additional margin of safety if they limited in their ability to infect humans especially when they are designed as replication-deficient vectors.

Adenoviruses as Vaccine Vectors

Adenoviral vectors have transitioned from tools for gene replacement therapy to bona fide vaccine delivery vehicles. They are attractive vaccine vectors as they induce both innate and adaptive immune responses in mammalian hosts. Adenoviral vectors have been tested to deliver as subunit vaccine systems for numerous infections infectious diseases, such as malaria, tuberculosis, Ebola and HIV-1. Additionally they have been explored as vaccines against different tumor associated antigens. Thus far most adenovirally vectored vaccines have been constructed as minimally modified adenoviral vectors of human and animal serotypes.

The dynamics of adenoviral gene expression have made the design of adenoviral packaging systems difficult: expression of the adenoviral early functional transcription region (E1A) gene induces expression of the adenoviral late genes (structural, immunogenic genes), which in turn kills the cell

Accordingly, a host cell that constitutively expresses the adenoviral early genes cannot carry the wildtype adenoviral late cistron. Previous host cells for propagating adenoviral vectors are not bonafide “packaging” cells, such as, but not limited to human cellsm the 293. QBI and PERC 6 cells that express only early (non-structural) adenoviral genes, not the adenoviral late genes needed for packaging. Adenoviral late also early genes have to be provided They previously been provided either by the minimally modified adenoviral vector in cis or by a helper adenovirus in trans.

The adenoviral genes found in minimally modified adenoviral vectors or in contaminating helper adenoviruses contribute to inflammatory and immune responses to the adenoviral vector preparation: decrease the immune response to a gene of interest of an adenoviral based vaccine, interferes with normal cellular functions, and to contamination in adenovirally based protein expression.

Adenoviral vectors have mostly been used for human therapy and as carriers of human vaccines. Both human as well as primate adenoviral vectors have proven highly efficient inducing broad humoral and cellular immune responses. Even though human adenoviral vector based vaccine have shown immunogenicity in animals, such as birds, they efficacy proved low requiring high doses and thus high costs. Therefore, it will be necessary to develop strategies to create adenoviral gene transfer vectors from a given animal species to be used in this species. This approach will lead to the production of highly efficient and potent vaccine carriers. Producing these vaccines as fully deleted “gutted” vaccine furthermore will focus the immune response and limit the induction of anti-adenoviral immune responses. Furthermore, fully deleted “gutted” adenoviral vector provide a large payload that will allow the delivery of several transgenes against several diseases. Therefore a single construct can be used as a basis of a broad combination vaccine.

### SUMMARY

A system of high capacity gene transfer vectors is being described for veterinary applications in animals of the class aves or birds. These vectors are based on aviadenoviruses (AAd). In one aspect, this invention describes the general design of these vectors as exemplified by, but not limited to, gene transfer vectors based on the CELO AAd. In another aspect, these vectors are based on other AAd viruses, such as but not limited to, AAd of the eight species and ten serotypes of AAd that have been identified. Non-limiting examples of such AAd viruses are the Fowl Aviadenovirus A, the Falcon Aviadenovirus A, the Quail Bronchitis Virus, the Egg Drop Syndrome virus, the hemorrhagic Enteritis virus, the Marble Spleen Disease Virus and the Inclusion Body Hepatitis Virus.

In one aspect, this invention describes the construction and use of partially as well as fully deleted AAd-based vectors that are designed as replication-defective vectors to enhance their capacity for the delivery of therapeutic transgenes, such as heterologous gene sequences, and to strengthen their safety profile. The use of such vectors as vaccine carriers is especially considered.

In one aspect, this invention is also based in part on the identification and genetic modification of host cells to be used for the replication and encapsidation of the different replication-deficient AAd vectors. In one aspect, this invention also describes the use of such AAd-based vectors to be used as gene transfer vectors in birds, but also in other animals including humans. Special consideration is given to the use such vectors for the development of vaccines.

In one aspect, the invention provides vaccine constructs that carry one or more transgene expression constructs in its genome. The vaccine constructs are designed as an expression cassette with a promoter, a transgene or a set of transgenes separated by an internal ribosomal entry site, and a poly-adenylation site. The transgenes can be derived from different infectious pathogens, such as, but not limited to, viruses, bacteria, protozoa, prions and nematodes. The transgenes can be coding for a protein or proteins whose expression are linked to malignant growths. This transgene sequence can be under the control of or operably linked to an adenoviral Major Late Promoter (MLP), an adenoviral tripartite leader (TPL) sequence, an adenoviral splice acceptor sequence, and/or an adenoviral poly-adenylation signal sequence. In certain embodiments, the transgene expression cassette comprises and/or is under the control of an non-adenoviral transcriptional and/or translational control sequence, such as an enhancer, promoter, intron sequence, and/or leader sequence from cytomegalovirus (CMV), rous sarcoma virus (RSV), or simian virus 40 (SV40), or any combination of such elements. In certain embodiments, the transgene sequence is modified to increase expression. For example, the transgene sequence can be codon optimized and/or modified to include a consensus Kozak sequence. In certain embodiments, the transgene sequence encodes an immunogenic polypeptide from an infectious pathogen, such as influenza virus, human papilloma virus (HPV), human immunodeficiency virus (HIV), Bacillus, Shigella, Mycobacterium, Plasmodium, etc. In certain embodiments, the transgene sequence encodes at least two separate polypeptides and/or a multimer of immunogenic epitopes from an infectious pathogen.

Other objects and features will be in part apparent and in part pointed out hereinafter.

### DESCRIPTION OF THE DRAWINGS

FIG. 1 is a diagrammatic representation of the genome of the CELO aviadenovirus and the human adenoviruses of the serotypes 2 and 5.

FIG. 2 is a diagrammatic representation of the genome of the CELO aviadenovirus and of CELO AAd-derived replication-competent and replication-deficient gene transfer vectors.

FIG. 3 is a diagrammatic representation of a complimentary genetic construct transfected into a host cell that enables the replication and production of partially deleted replication-deficient CELO AAd-derived vectors.

FIG. 4 is a diagrammatic representation of CELO packaging expression plasmids enabling the replication and production of CELO AAd-derived fully deleted “gutted” vectors.

FIG. 5 is a diagrammatic representation of the construction of adenoviral and CELO genome fragments to enhance the function of packaging or host cells.

FIG. 6 is a diagrammatic representation of the replication and packaging of a fully deleted “gutted” CELO derived aviadenoviral vector.

### DETAILED DESCRIPTION

As used herein, the following terms shall have the following meanings.

The term “adenoviral vector” refers to a wild-type, mutant, and/or recombinant adenoviral genome, as well as adenoviruses comprising such a genome. An adenoviral vector can comprise all or part of the genome of any adenoviral serotype, as well as combinations thereof (i.e., hybrid genomes).

The term “aviadenoviral vector” refers to an adenoviral vector derived from an adenovirus preferentially found in animals of the class aves or birds.

The term “infectious pathogen” refers to any agent capable of infection and causing deterioration in health and/or triggering an immune response. In certain embodiments, the infectious pathogen is a virus, such as an influenza virus, retrovirus (e.g., HIV, Rous Sarcoma Virus (RSV), human endogenous retrovirus K (HERV-K)), human endogenous retrovirus K (HERV-K), papillomavirus (e.g., human papilloma virus), picornavirus (e.g., Hepatitis A, Poliovirus), hepadnavirus (e.g., Hepatitis B), flavivirus (e.g., Hepatitis C, Yellow Fever virus, Dengue Fever virus, Japanese encephalitis virus, West Nile virus), togavirus (e.g., chikungunya virus, Eastern equine encephalitis (EEE) virus, Western equine encephalitis (WEE) virus, Venezuelan equine encephalitis (VEE) virus), herpesvirus (e.g., Cytomegalovirus), paramyxovirus (Parainfluenza virus, Pneumonia virus, Bronchiolitis virus, common cold virus, Measles virus, Mumps virus), rhabdovirus (e.g., Rabies virus), Filovirus (e.g., Ebola virus), bunyavirus (e.g., Hantavirus, Rift Valley Fever virus), calicivirus (e.g., Norovirus), or reovirus (e.g., Rotavirus, Epstein-Barr virus, Herpes simplex virus types 1 & 2).

In other embodiments, the infectious pathogen is a prokaryotic organism such as a gram-negative bacterium, gram-positive bacterium, or other type of bacterium. Such prokaryotic organisms include, but are not limited to, Bacillus (e.g., Bacillus anthracis), Mycobacterium (e.g., Mycobacterium tuberculosis, Mycobacterium Leprae), Shigella (e.g., Shigella sonnei, Shigella dysenteriae, Shigella flexneri), Helicobacter (e.g., Helicobacter pylori), Salmonella (e.g., Salmonella enterica, Salmonella typhi, Salmonella typhimurium), Neisseria (e.g., Neisseria gonorrhoeae, Neisseria meningitidis), Moraxella (e.g., Moraxella catarrhalis), Haemophilus (e.g., Haemophilus influenzae), Klebsiella (e.g., Klebsiella pneumoniae), Legionella (e.g., Legionella pneumophila), Pseudomonas (e.g., Pseudomonas aeruginosa), Acinetobacter (e.g., Acinetobacter baumannii), Listeria (e.g., Listeria monocytogenes), Staphylococcus (e.g., methicillin-resistant, multidrug-resistant, or oxacillin-resistant Staphylococcus aureus), Streptococcus (e.g., Streptococcus pneumoniae, Streptococcus pyogenes, Streptococcus agalactiae), Corynebacterium (e.g., Corynebacterium diphtheria), Clostridium (e.g., Clostridium botulinum, Clostridium tetani, Clostridium difficile), Chlamydia (e.g., Chlamydia pneumonia, Chlamydia trachomatis), Camphylobacter (e.g., Camphylobacter jejuni), Bordetella (e.g., Bordetella pertussis), Enterococcus (e.g., Enterococcus faecalis, Enterococcus faecum, Vancomycin-resistant enterococcus (VRE)), Vibrio (e.g., Vibrio cholerae), Yersinia (e.g., Yersinia pestis), Burkholderia (e.g., Burkholderia cepacia complex), Coxiella (e.g., Coxiella burnetti), Francisella (e.g., Francisella tularensis), and Escherichia (e.g., enterotoxigenic, enterohemorrhagic or Shiga toxin-producing E. coli, such as ETEC, EHEC, EPEC, EIEC, and EAEC)).

In still other embodiments, the infectious pathogen is a eukaryotic organism. Examples of eukaryotic organisms include, but are not limited to protists, such as a Plasmodium (e.g., Plasmodium falciparum, Plasmodium vivax, Plasmodium ovale, Plasmodium malariae Plasmodium diarrhea), and fungi such as Candida (e.g., Candida albicans), Aspergillus (e.g., Aspergillus fumigatus), Cryptococcus (e.g., Cryptococcus neoformans), Histoplasma (e.g., Histoplasma capsulatum), Pneumocystis (e.g., Pneumocystis jirovecii), and Coccidioides (e.g., Coccidioides immitis).

The term “cancer”, “cancerous”, “malignancy” and “malignant” refer to medical conditions characterized by abnormal increases in the proliferation of particular population of cells. The cancerous cells can be derived from any tissue or organ including, e.g., skin, muscle, lung, heart, liver, kidney, neural tissue, etc. In certain embodiments, the cancer is benign (e.g., a benign tumor). In other embodiments, the cancer is malignant (e.g., a malignant tumor). In certain embodiments, the cancer is metastatic (i.e., the cancer cells are able to migrate from their place of origin to another tissue or organ).

Additional terms shall be defined, as needed, throughout the specification.

The present invention is directed to recombinant adenoviral vaccines. The invention is based, in part, on the development of novel recombinant adenoviral vectors that express heterologous sequences or transgenes at high levels. The invention is also based, in part, on the development of novel recombinant adenoviral vectors designed to improve host immune response and circumvent pre-existing neutralizing antibodies. The invention is also based, in part, on the development of novel recombinant adenoviral vectors to be used as antigen-specific and/or universal influenza vaccines.

Accordingly, in one aspect, the invention provides an adenoviral vector comprising a transgene sequence. As used herein, a “transgene sequence” is a nucleic acid sequence that, upon integration into an adenoviral vector, creates a non-naturally occurring juxtaposition of adenoviral sequences with the nucleic acid sequence. Typically, a transgene sequence will comprise nucleic acid sequence that is non-adenoviral in origin. For example, the transgene sequence can be entirely, mostly, or partially non-adenoviral (e.g., a mosaic of adenoviral and non-adenoviral sequences) in origin. In some instances, however, a transgene sequence can be entirely adenoviral in origin, e.g., an adenoviral sequence from one type of adenovirus can be integrated into an adenoviral vector generated from a different type of adenovirus. For instance, an adenoviral sequence encoding a hexon or fiber protein from one type of adenovirus can be integrated into an adenoviral vector generated from a different type of adenovirus to produce recombinant adenovirus with fiber proteins from different serotypes and/or adenovirus with chimeric hexon and fiber proteins. Adenoviral vectors comprising a transgene sequence can be useful, e.g., as vaccines against infectious pathogens or cancerous cells. Thus, the transgene sequence can encode an antigen from an infectious pathogen. Alternatively, the transgene sequence can encode an antigen associated with cancerous cells.

In certain embodiments, the transgene sequence encodes all or part of a protein produced by an infectious pathogen. The protein, or fragment thereof (e.g., cleavage product, structural domain, unit(s) of secondary structure, B-cell epitope, cytotoxic T lymphocyte (CTL) epitope, helper T lymphocyte (HTL) epitope, etc.), can be located on the surface of the infectious pathogen. For example, the protein or fragment thereof can be highly antigenic, involved in cellular targeting, and/or involved in cellular entry. Alternatively, the protein, or fragment thereof (e.g., cleavage product, structural domain, unit(s) of secondary structure, HTL or CTL epitope, etc.), can be located internal to the infectious pathogen. For example, the protein or fragment thereof can be an intracellular protein, a capsid or core protein of an enveloped virus, a core protein of a non-enveloped virus, etc.

In certain embodiments, the epitope, structural domain, or unit of secondary structure is evolutionarily conserved. As used herein, the term “evolutionarily conserved” means that a sequence is at least about 50% conserved among a diverse set of strains of a particular infectious pathogen. For viruses, a diverse set of strains includes at least one isolate from each identified subclassification (e.g., serotype) capable of infecting and thereby causing disease or illness in the target population for the vaccine, or a representative number of infectious isolates encompassing the known diversity in such strains. For example, in certain embodiments, a diverse set of influenza strains includes representative strains that are associated with disease in man, swine, and/or birds, including H1N1 strains (e.g., A/Wilson-Smith/33, A/New Calcdonia/20/99, A/Swine Korea/S10/2004, A/Brevig Mission/1/1918, A/Pureto Rico/8/34/Mount Sinai, A/California/7/2009, A/California/05/2009, A/California/08/2009, A/Texas/04/2009, A/swine/Saskatchewan/18789/02, A/mallard/Alberta/130/2003, A/mallard/Alberta/2001, A/swine/Cotes d'Armor/1482/99, A/swine/Betzig/2/2001, and/or A/turkey/Germany/3/91), H3N2 strains (e.g., A/Perth/16/2009), H2N2 strains (e.g., A/Japan/305/57, A/Ann Arbor/6/60, A/Canada/720/05, A/mallard/NY/6750/78, A/mallard/Potsdam/177-4/83, and/or A/duck/Hokkaido/95/2001), N3N2 strains (e.g., A/Hong Kong/1/66, A/Charlottesville/03/2004, A/Canterbury/129/2005, A/Fujian/411/01-like, A/duck/Korea/S9/2003, A/swine/Texas/4199-2/98, A/turkey/Ohio/313053/2004, and/or A/turkey/North Carolina/12344/03), H5N1 strains (e.g., A/swine/Shandong/2/03, A/goose/Guangdong/1/96, A/duck/Hunan/114/05, A/VietNam/1203/2004, A/VietNam/DT-036/2005, A/Vietnam/i194/2004, A/Vietnam/1203/2004, A/Anhui/1/2005, A/Egypt/2321/2007, A/Egypt/3300-NAMRU3/2008, A/grebe/Novosibirsk/29/2005, A/Bar-headed goose/Mondolia/1/05, A/cat/Thailand/KU-02/04, A/Hong Kong/213/03, A/chicken/Guangdong/174/04, and/or A/HK/159/97), H6N1 strains (e.g., A/teal/Hong Kong/1073/99), H6N2 strains (e.g., A/chicken/California/0139/2001, and/or A/guillemot/Sweden/3/2000), H6N9 strains (e.g., A/goose/Hong Kong/W217/97), H7N1 strains (e.g., A/FPV/Rostock/34), H7N3 strains (e.g., A/chicken/British Columbia/04, and/or A/turkey/Italy/220158/2002), H7N7 strains (e.g., A/chicken/Netherlands/1/2003, A/Netherlands/219/03, A/FPV/Dobson/27, and/or A/chicken/FPV/Weybridge), H9N2 strains (e.g., A/shorebird/Delaware/9/96, A/swine/Korea/S452/2004, A/duck/Hong Kong/Y439/97, A/Hong Kong/1073/99, A/HK/2108/2003, A/quail/Hong Kong/G1/97, A/duck/Hong Kong/Y280/97, A/chicken HK/FY23/03, and/or A/chicken HK/G9/97), and B influenza strains (e.g., B/Brisbane/60/2008). In certain embodiments, a diverse set of influenza strains includes all of the foregoing strains as well as additional influenza strains known to be associated with disease in man, swine, or birds. For cellular pathogens, such as bacteria, protists, fungi, etc., a diverse set of strains includes at least one isolate from each species capable of infecting and thereby causing disease or illness in the target population for the vaccine, or a representative number of infectious isolates encompassing the know diversity in such strains. In certain embodiments, the epitope and/or structural motif is at least 60%, 70%, 75%, 80%, 85%, 90%, 95%, or more conserved.

In certain embodiments, the transgene sequence encodes an antigen from an influenza virus. A suitable influenza antigen can be a surface antigen, such as hemagglutinin (HA), neuraminidase (NA), M2, or a fragment thereof (e.g., one or more HTL or CTL epitopes). Other suitable influenza antigens include M1, NP, NS1, NS2, PA, PB1, and PB2, or fragments thereof (e.g., one or more HTL or CTL epitopes).

The transgene sequence can encode an immunogenic protein or antigen from any infectious pathogen disclosed herein. For instance, in some embodiments, the transgene sequence encodes an immunogenic protein from a virus, a bacterium, a protist, and/or a fungus. In one embodiment, the transgene sequence encodes an immunogenic protein from influenza virus, poliovirus, human immunodeficiency virus (HIV), human papilloma virus (HPV), chikungunya virus, and/or Dengue Fever virus. In another embodiment, the transgene sequence encodes an immunogenic protein from Bacillus (e.g., Bacillus anthracis), Mycobacterium (e.g., Mycobacterium tuberculosis, Mycobacterium Leprae), Shigella (e.g., Shigella sonnei, Shigella dysenteriae, Shigella flexneri), Streptococcus, and/or Escherichia (e.g., enterotoxigenic, enterohemorrhagic or Shiga toxin-producing E. coli). In another embodiment, the transgene sequence encodes an immunogenic protein from enterotoxigenic E. coli (ETEC), enteropathogenic E. coli (EPEC), enteroinvasive E. coli (EIEC), enterohemorrhagic E. coli (EHEC), and/or enteroaggregative E. coli (EAEC). In still another embodiment, the transgene sequence encodes an immunogenic protein from Burkholderia (e.g., Burkholderia cepacia complex), Pseudomonas (e.g., Pseudomonas aeruginosa), Clostridium (e.g., Clostridium botulinum, Clostridium tetani, Clostridium difficile), Staphylococcus (e.g., methicillin resistant, multidrug resistant, or oxacillin resistant Staphylococcus aureus), Enterococcus (e.g., Enterococcus faecalis, Enterococcus faecum, Vancomycin-resistant enterococcus (VRE)), Streptococcus (e.g., Streptococcus pneumoniae, Streptococcus pyogenes, Streptococcus agalactiae), and/or Vibrio (e.g., Vibrio cholerae). In another embodiment, the transgene sequence encodes an immunogenic protein from Camphylobacter (e.g., Camphylobacter jejuni), Bordetella (e.g., Bordetella pertussis), Chlamydia (e.g., Chlamydia pneumonia, Chlamydia trachomatis), Corynebacterium (e.g., Corynebacterium diphtheria), Legionella (e.g., Legionella pneumophila), Listeria (e.g., Listeria monocytogenes), Neisseria (e.g., Neisseria gonorrhoeae, Neisseria meningitidis), Salmonella (e.g., Salmonella enterica, Salmonella typhi, Salmonella typhimurium), Yersinia (e.g., Yersinia pestis), Haemophilus (e.g., Haemophilus influenzae), Helicobacter (e.g., Helicobacter pylori), Coxiella (e.g., Coxiella burnetti), and/or Francisella (e.g., Francisella tularensis). In certain embodiments, the transgene sequence encodes an immunogenic protein from influenza, HIV, HPV, Bacillus anthracis, Plasmodium and/or Shigella. In still other embodiments, the transgene sequence encodes an immunogenic protein from influenza, HIV, and/or Bacillus anthracis.

Influenza antigens encoded by the transgene sequence can be from any influenza strain, presently existing or subsequently isolated, including, e.g., a strain associated with the Spanish flu of 1918 (H1N1), the Asian flu of 1957 (H2N2), the Hong Kong flu of 1968 (H3N2), the Hong Kong flu of 1997 (H5N1), the Vietnam flu of 2004 (H5N1), the swine flu of 2009 (H1N1) etc. Thus, for example, the HA antigen can be an H1, H2, H3, H4, H5, H6, H7, H8, H9, H10, H11, H12, H13, H14, H15, H16, or B HA antigen, while the NA antigen can, for example, be an N1, N2, N3, N4, N5, N6, N7, N8, or N9 NA antigen. In some embodiments, the HA antigen is an H1, H3, H5, or B HA antigen. Non-limiting examples of influenza strains that can be the basis for a heterologous sequence of the invention include: A/goose/Guangdong/1/96 (H5N1); A/Brevig Mission/1/1918 (H1N1); A/Wilson-Smith/33 (H1N1); A/Puerto Rico/8/34/Mount Sinai (H1N1); A/Fort Monmouth/1/47 (H1N1); A/USSR/90/1977 (H1N1); A/New Calcdonia/20/1999 (H1N1); A/Solomon Islands/3/2006 (H1N1); A/Brisbane/59/2007 (H1N1); A/California/7/2009 (H1N1); A/California/14/2009 (H1N1); A/California/08/2009 (H1N1); A/California/05/2009 (H1N1); A/Texas/04/2009 (H1N1); A/Mexico/InDRE4114/2009 (H1N1); A/New York/1669/2009 (H1N1); A/Canada-AB/RV1532/2009 (H1N1); A/Leningrad/134/47/57 (H2N2); A/Ann Arbor/6/60 (H2N2); A/Berlin/3/64 (H2N2); A/Tokyo/3/67 (H2N2); A/Singapore/1/57 (H2N2); A/Hong Kong/1/68 (H3N2); A/Albany/1/76 (H3N2); A/Panama/2007/99 (H3N2); A/Wisconsin/67/05 (H3N2); A/Hong Kong/1774/99 (H3N2); A/Moscow/10/99 (H3N2); A/Hiroshima/52/2005 (H3N2); A/California/7/2004 (H3N2); A/New York/55/2004 (H3N2); A/Brisbane/10/2007 (H3N2); A/Perth/16/2009 (H3N2); A/goose/Guiyang/337/2006 (H5N1) Glade 4; A/HK/156/97 (H5N1); A/HK/483/97 (H5N1); A/VietNam/i194/2004 (H5N1) Glade 1; A/VietNam/1203/2004 (H5N1) Glade 1; A/duck/NCVD1/07 (H5N1); A/chicken/VietNam/NCVD-21/07 (H5N1); A/Indonesia/5/05 (H5N1) Glade 2.1; A/Turkey/65-596/06 (H5N1) Glade 2.2; A/chicken/India/NIV33487/2006 (H5N1) Glade 2.2; A/turkey/Turkey/1/2005 (H5N1) Glade 2.2; A/Egypt/902782/2006 (H5N1); A/Egypt/2321/2007 (H5N1); A/Egypt/3300-NAMRU3/2008 (H5N1); A/Anhui/1/2005 (H5N1); A/China/GD01/2006 (H5N1); A/common magpie/Hong Kong/50525/07 (H5N1) Glade 2.3.2; A/Japanese white-eye/Hong Kong/1038/2006 (H5N1) Glade 2.3.4; A/chicken/VietNam/NCVD-15/2007 (H5N1); A/chicken/Italy/2335/2000 (H7N1); A/turkey/Italy/3675/99 (H7N1); A/chicken/New York/21211-2/05 (H7N2); A/New York/107/03 (H7N2); A/chicken/British Columbia/GSC human B/04 (H7N3); A/Canada/rv504/04 (H7N3); A/chicken/British Columbia/CN-6/04 (H7N3); A/equine/San Paulo/4/76 (H7N7); A/seal/Mass/1/1980 (H7N7); A/chicken/Victoria/1/1985 (H7N7); A/chicken/Netherlands/2586/2003 (H7N7); A/mallard/California/HKWF1971/2007 (H7N7); A/chicken/Beijing/1/94 (H9N2); A/quail/Hong Kong/G1/1997 (H9N2); A/Korea/KBNP-0028/2000 (H9N2); A/chicken/Hong Kong/G9/97 (H9N2); A/chicken/Hong Kong/CSW153/2003 (H9N2); A/chicken/Shantou/6781/2005 (H9N2); A/chicken/Jiangsu/L1/2004 (H9N2); A/Hong Kong/1073/99 (H9N2); A/Hong Kong/2108/2003 (H9N2); A/chicken/Shiraz/AIV-IR004/2007 (H9N2); A/chicken/Zibo/L2/2008 (H9N2); A/chicken/Henan/L1/2008 (H9N2); A/avian/Israel/313/2008 (H9N2) and B/Brisbane/60/2008. Additional influenza strains can be readily identified by persons skilled in the art.

In certain embodiments, the transgene sequence encodes an influenza HA antigen selected from H1, H3, H5, or B influenza virus. The HA antigen may, in some embodiments, be derived from one or more of the strains selected from the group consisting of A/Vietnam/i194/2004, A/Vietnam/1203/2004, A/Anhui/1/2005, A/Egypt/2321/2007, A/Egypt/3300-NAMRU3/2008, A/Perth/16/2009, A/California/05/2009, or B/Brisbane/60/2008. In some embodiments, the transgene sequence encodes an influenza NP or M1 antigen. In one embodiment, the NP or M1 antigen is derived from A/Texas/04/2009 or A/California/08/2009 influenza strains.

In other embodiments, the transgene sequence encodes an antigen from human papilloma virus (HPV). The HPV can be of any known or later discovered strain (e.g., HPV-1, HPV-2, HPV-6, HPV-11, HPV-16, HPV-18, HPV-31, HPV-45, etc.). In one embodiment, the transgene sequence encodes an antigen from a HPV-16 or HPV-18 strain. In certain embodiments, the HPV antigen is a surface antigen, such as full-length L1 protein or a fragment thereof (e.g., an evolutionarily conserved epitope and/or a HTL or CTL epitope). In one embodiment, the transgene sequence encodes a full-length L1 protein that is fully or partially codon-optimized. In other embodiments, the HPV antigen is full-length L2 or a fragment thereof (e.g., a evolutionarily conserved epitope and/or a HTL or CTL epitope). In other embodiments, the HPV antigen is a L1 hybrid polypeptide or a L1/L2 hybrid polypeptide. For instance, in one particular embodiment, the HPV antigen is a L1 polypeptide comprising a fragment of the L2 polypeptide (e.g., an L2 fragment can be inserted into a loop of the L1 polypeptide). In still other embodiments, the HPV antigen is a full-length E6 or E7 protein, or a fragment thereof (e.g., an evolutionarily conserved epitope and/or a HTL or CTL epitope). In still other embodiments, the HPV antigen is a fusion protein comprising L1, L2 and/or E6 and E7 proteins. For example, in some embodiments, the HPV antigen is a fusion protein comprising a L1/L2 hybrid polypeptide fused to an E7 protein. In other embodiments, the HPV antigen is a fusion protein comprising a L1/L2 hybrid polypeptide fused to an E6 protein.

In other embodiments, the transgene sequence encodes an antigen from human immunodeficiency virus (HIV). The HIV can be of any known or later discovered strain (e.g., HIV-1, HIV-2, etc.). In certain embodiments, the HIV antigen is a surface antigen, such as full-length Env protein (e.g., gp160) or a fragment or oligomer thereof (e.g., gp140, gp120, gp41, an evolutionarily conserved epitope, and/or a HTL or CTL epitope). In other embodiments, the HIV antigen is a full-length capsid protein (p24), matrix protein (p17), or a fragment thereof (e.g., a evolutionarily conserved epitope and/or a HTL or CTL epitope). In other embodiments, the HIV antigen is a Tat (e.g., p16 or p14), Rev (p19), Vif (p23), Vpr (p14), Nef (p27), Vpu (p16), or Gag protein. The HIV antigen can be any HIV protein, full-length or otherwise, such as a HTL or CTL epitope, and can be any evolutionarily conserved sequence. In some embodiments, the HIV antigen sequence can be engineered to contain heterologous trimerization domains (e.g., from yeast GCN, such as from GCN4, and T4 bacteriophage fibritin-FT motifs) or certain signal sequences for post-translational modifications, such as glycosylphosphatidylinisotol (GPI) anchor sites. For instance, in one embodiment, an HIV envelope protein, such as gp140 or gp120, can be modified to contain a GPI anchor site. In another embodiment, an HIV gp140 sequence can be modified to contain a heterologous GCN trimerization domain and/or a GPI anchor site. In some embodiments, the GCN trimerization domain or GPI anchor site is fused to the carboxyl terminus of an HIV envelope protein sequence (e.g., HIV gp140 sequence).

In other embodiments, the transgene sequence encodes an antigen from a Bacillus bacterium. The Bacillus can be any of a number of pathogenic species (e.g., B. anthracis, B. cereus, etc.) and can be any known or later discovered isolate of such a species. In certain embodiments, the Bacillus antigen is a surface antigen, such as protein resident in the cellular membrane, or a fragment thereof (e.g., an evolutionarily conserved epitope, and/or a HTL or CTL epitope). In other embodiments, the Bacillus antigen is an intracellular protein or a fragment thereof (e.g., an evolutionarily conserved epitope, and/or a HTL or CTL epitope). In certain embodiments, the Bacillus antigen is associated with host cell entry. For example, the antigen can be a target cell-binding protein (e.g., protective antigen (PrAg or PA)), a metallopeptidase (e.g., lethal factor (LF)), an adenylate cyclase (e.g., edema factor (EF)), or fragment thereof (e.g., an evolutionarily conserved epitope, and/or a HTL or CTL epitope). In some embodiments, the Bacillus antigen can be modified to delete a thermolysin cleavage site or contain a GPI anchor. In one embodiment, the transgene sequence encodes protective antigen or a modified protective antigen which has been modified to remove a thermolysin cleavage site or contain a GPI anchor.

In other embodiments, the transgene sequence encodes an antigen from a Shigella bacterium. The Shigella can be any of a number of pathogenic species (e.g., S. sonnei, S. dysenteriae, S. flexneri, etc.) and can be any known or later discovered isolate of such a species. In certain embodiments, the Shigella antigen is a surface antigen, such as protein resident in or associated with the cellular membrane, such as an integral membrane protein or a peripheral membrane protein, or a fragment thereof (e.g., an evolutionarily conserved epitope, and/or a HTL or CTL epitope). For example, the antigen can be an outer membrane protein, such as Karp strain p56. In other embodiments, the Shigella antigen is an intracellular protein or a fragment thereof (e.g., an evolutionarily conserved epitope, and/or a HTL or CTL epitope). In certain embodiments, the Shigella antigen is associated with host cell entry, such as invasion proteins IpaB, IpaC, or IpaD protein. In another embodiment, the Shigella antigens are universal antigens comprising IcsP and/or SigA polypeptides.

In other embodiments, the transgene sequence encodes an antigen from a Mycobacterium. The Mycobacterium can be any of a number of pathogenic species (e.g., M. tuberculosis, M. leprae, M. lepromatosis, etc.) and can be any known or later discovered isolate of such a species. In certain embodiments, the Mycobacterium antigen is a surface antigen, such as protein resident in or associated with the cellular membrane, such as an integral membrane protein or a peripheral membrane protein, or a fragment thereof (e.g., an evolutionarily conserved epitope, and/or a HTL or CTL epitope). In other embodiments, the Mycobacterium antigen is an intracellular protein or a fragment thereof (e.g., an evolutionarily conserved epitope, and/or a HTL or CTL epitope). In certain embodiments, the Mycobacterium antigen is selected from the group consisting of Ag85A, Ag85B, Ag85C, ESAT-6, CFP-10, HspX, and combinations thereof.

In other embodiments, the transgene sequence encodes an antigen from a Plasmodium. The Plasmodium can be any of a number of pathogenic species (e.g., P. falciparum, P. vivax, P. ovale, P. malariae, etc.) and can be any known or later discovered isolate of such a species. In certain embodiments, the Plasmodium antigen is a surface antigen, such as protein resident in or associated with the cellular membrane, such as an integral membrane protein or a peripheral membrane protein, or a fragment thereof (e.g., an evolutionarily conserved epitope, and/or a HTL or CTL epitope). In other embodiments, the Plasmodium antigen is an intracellular protein or a fragment thereof (e.g., an evolutionarily conserved epitope, and/or a HTL or CTL epitope). In certain embodiments, the Plasmodium antigen is selected from the group consisting of CS, CSP (uncleaved), MSP1, MSP2 (c-terminal p42), LSA1, EBA-175, AMA1, FMP1, Pfs48/45, and MSPS.

In certain embodiments, the transgene sequence encodes an antigen from Streptococcus pneumoniae (e.g. Pneumococcus). In certain embodiments, the Streptococcus pneumoniae antigen is a surface antigen, such as protein resident in or associated with the cellular membrane, such as an integral membrane protein or a peripheral membrane protein, or a fragment thereof (e.g., an evolutionarily conserved epitope, and/or a HTL or CTL epitope). In other embodiments, the Streptococcus pneumoniae antigen is an intracellular protein or a fragment thereof (e.g., an evolutionarily conserved epitope, and/or a HTL or CTL epitope). In certain embodiments, the Streptococcus pneumoniae antigen is selected from the group consisting of pneumococcal surface proteins (e.g., PspA, PspC), pneumolysin (Ply), neuraminidase enzymes (e.g., NanA, NanB), autolysin A (LytA), pneumococcal histidine-triad proteins, PiaA, PiuA, fructose-bisphosphate aldolase (FBA), adhesin A, and pneumolysoid.

In still other embodiments, the transgene sequence encodes a surface antigen, internal protein, toxin, invasion-associated protein, protease or other enzymes, heat shock protein, or other antigen from any other infectious pathogen. For example, the surface antigen can be from an infectious pathogen selected from the group consisting of Bordetalla pertussis, Chlamydia pneumonia (e.g., membrane protein D, outer membrane protein), Chlamydia trachomatis (e.g., membrane protein D, outer membrane protein), Legionella pneumophilia, Staphylococcus aureus, including methicillin-resistant, multi-drug-resistant, and oxacillin-resistant strains (e.g., IsdA, IsdB, SdrD, SdrE), Streptococcus pneumoniae (e.g., PsPA), Streptococcus aeruginosa (e.g., flagellar Ag, porins), Streptococcus pyogenes (e.g., M protein, Fibronectin-binding protein Sfb1), Streptococcus agalactiae, Enterohemorrhagic E. coli (e.g., Intimin, FimH adhesin), Haemophilis influenzae (e.g., Pili, P1, P2, P4, P6), Candida (e.g., A1s1p, Als3p), Coccidioides immitis (e.g., Ag2), Pseudomonas aeruginosa (e.g., flagellar antigen, porins), Rous sarcoma virus (e.g., F protein, G protein), human endogenous retrovirus K (e.g., melanoma antigen HERV-K-MEL), herpes virus (e.g., glycoprotein D2), Dengue Fever virus (e.g., DEN1, DEN2, DEN3, DEN4 envelope proteins, tetravalent 4.times. EDIII domain protein), etc. The toxin can be selected from the group consisting of labile toxin of Camphylobacter jejuni, Toxins A and B of Clostridium difficile, pyrogenic exotoxins and endotoxins from Streptococcus pyogenes, Toxin B of Vibrio cholerae, Shiga toxin (e.g., Stx-1, Stx-2) of enterohemorrhagic E. coli, the exotoxin A from Pseudomonas aeruginosa etc. The protease or other enzymes can be selected from the group consisting of secreted protease factor of Chlamydia, pneumolysin, autolysin, or neuraminidase of Streptococcus pneumoniae, cystein protease or C5a peptidase from Streptococcus pyogenes, urease from Helicobacter pylori, urease of Coccidioides immitis, His-62, H antigen, and hsp70 of Histoplasma capsulatum, etc.

In certain embodiments, the transgene sequence encodes all or part of a protein produced by a cancer cell. The protein, or fragment thereof (e.g., cleavage product, structural domain, unit(s) of secondary structure, B-cell epitope, cytotoxic T lymphocyte (CTL) epitope, helper T lymphocyte (HTL) epitope, etc.), can be located on the surface of the cancer cell. For example, the protein or fragment thereof can be highly antigenic and/or a marker for the cancer cell (e.g., a cancer cell-specific marker or an antigen highly enriched on the cancer cells). Alternatively, the protein, or fragment thereof (e.g., cleavage product, structural domain, unit(s) of secondary structure, HTL or CTL epitope, etc.), can be located internal to the cancer cell. For example, the protein or fragment thereof can be a cytosolic protein, a nuclear protein, etc.

In certain embodiments, the transgene sequence comprises at least one complete open reading frame (ORF), wherein the at least one complete ORF encodes a discrete polypeptide capable of being expressed in a host cell infected by the adenoviral vector. In certain embodiments, the transgene sequence comprises two or more complete ORFs, each of which encodes a discrete polypeptide capable of being expressed in a host cell infected by the adenoviral vector. One or more of the discrete polypeptides can be a full-length protein or fragment thereof, as described above. Likewise, one or more of the discrete polypeptides can be a multimer of protein domains, structural motifs, or epitopes (e.g., B-cell, HTL or CTL epitopes), as described above. For example, in certain embodiments, the transgene sequence comprises a first ORF that encodes a full-length protein (e.g., influenza HA) and a second ORF that encodes a multimer of protein domains, structural motifs, or epitopes (e.g., a multimer of one or more influenza M2 sequences, a multimer of one or more influenza B-cell epitopes, a multimer of one or more influenza HTL epitopes, or a multimer of one or more influenza CTL epitopes.

Thus, in some embodiments, the transgene sequence encodes a fusion protein. The fusion protein can comprise one or more epitopes or fragments from antigenic proteins or full-length proteins from the same infectious pathogen or a different infectious pathogen. For instance, in one embodiment, the fusion protein comprises a L1/L2 hybrid polypeptide of HPV as described herein fused to the E6 or E7 proteins of HPV. In some embodiments, the fusion protein comprises a L1/L2 hybrid polypeptide derived from HPV-16 (e.g., full length HPV-16 L1 protein with a HPV-16 L2 fragment inserted into a L1 loop) fused to an E7 protein. In other embodiments, the fusion protein comprises a L1/L2 hybrid polypeptide derived from HPV-18 (e.g., full length HPV-18 L1 protein with a HPV-18 L2 fragment inserted into a L1 loop) fused to an E6 protein. In another embodiment, the fusion protein comprises immunogenic fragments from influenza HA and NA proteins fused together (e.g., neutralization epitopes of influenza HA or NA proteins as described herein). In another embodiment, the fusion protein comprises one or more neutralization epitopes of influenza HA proteins as described herein fused to full-length influenza NA proteins. In still another embodiment, the fusion protein can be a multimer of various epitopes as described herein. For instance, the fusion protein can be a multimer of HTL epitopes, wherein each epitope is connected by a linker sequence (see Example 13 for a representative multimer). In some embodiments, the fusion protein encoded by the transgene sequence comprises an antigen from two or more species or serotypes of an infectious pathogen. For instance, the fusion protein can comprise EDIII domains from the envelope proteins from each of the four Dengue Fever virus serotypes 1-4.

In certain embodiments, the transgene sequence comprises two complete ORFs, wherein the first and second ORFs are oriented in parallel (e.g., head to tail). In certain related embodiments, the transgene sequence further comprises an internal ribosomal entry sequence (IRES) located 3′ to the stop codon of the first ORF and 5′ to the start codon of the second ORF, thereby allowing the polypeptides encoded by the first and second ORFs to be translated from a single mRNA transcript. Persons skilled in the art can readily identify suitable IRES sequences that are functional in mammalian (e.g., human) cells and how such sequences should be positioned to ensure sufficient translation of the second ORF.

In certain related embodiments, the transgene sequence comprises two complete ORFs, wherein the first and second ORFs are oriented in parallel (e.g., head to tail), and further comprises a splice acceptor located 3′ to the stop codon of the first ORF and 5′ to the start codon of the second ORF, thereby allowing the polypeptides encoded by the first and second ORFs to be translated from a single mRNA transcript or as two separate mRNA transcripts. Persons skilled in the art can identify splicing elements and incorporate them in the correct fashion. Splicing acceptors can be either consensus sequences (such as SV40 splice sites) or non-consensus sequences (such as the Ad5 ADP splice acceptor), depending upon the desired outcome. For example, in the adenovirus major late transcription unit, 3′ splice sites having atypical polypyrimidine tracts are preferred late in viral infection. See, e.g., Muhlemann et al. (1995), J. Virology 69(11):7324.

In certain related embodiments, the transgene sequence comprises two complete ORFs, wherein the first and second ORFs are oriented in parallel (e.g., head to tail), and further comprises a 2A skipping element (intra-ribosomal self-processing) located in frame between the 3′ end of the first ORF (stop codon removed) and 5′ in frame to the start codon of the second ORF, thereby allowing the polypeptides encoded by the first and second ORFs to be translated from a single mRNA transcript as a single peptide that “skips” a peptide bond at the location of the A2 element and thereby generates two polypeptides. Persons skilled in the art can identify 2A skipping elements such those derived from the foot and mouth disease virus (FMDV) and picornavirus, and organize them such that the two ORFs form a single continuous peptide.

In certain embodiments, the transgene sequence comprises two complete ORFs, wherein the first and second ORFs are oriented end-to-end. For example, the 3′ end of the first ORF can be adjacent to the 3′ end of the second ORF. Alternatively, the 5′ end of the first ORF can be adjacent to the 5′ end of the second ORF.

In general, the transgene sequence is part of a transcriptional unit that minimally contains a transcriptional enhancer and/or promoter and a poly adenylation sequence. In certain embodiments, the transcriptional unit further comprises one or more introns, one or more splice enhancers, a leader sequence, a consensus Kozak sequence, one or more elements that increase RNA stability and/or processing, or any combination thereof.

In certain embodiments, the transgene sequence is under the control of or operably linked to an adenoviral transcriptional and/or translational control sequence. As used herein in this context, “under the control of” and “operably linked to” mean that the transcription and/or translation of an ORF contained in a heterologous sequence is affected by the control sequence. Thus, for example, the transcription and/or translation of the ORF can be increased as a result of the adenoviral transcriptional and/or translational control sequence. In certain embodiments, “operably linked to” indicates that the control sequence and the heterologous sequence are in close proximity to one another. For example, in certain embodiments, an adenoviral control sequence that is operably linked to a heterologous sequence is located within about 100 bps, between about 100 and about 200 bps, between about 200 and about 300 bps, between about 300 and about 400 bps, or between about 400 and about 500 bps from one end of the heterologous sequence.

As used herein, an “adenoviral transcriptional and/or translational control sequence” is a nucleic acid sequence involved in transcriptional and/or translational regulation that is derived from an adenovirus. Such sequences include, but are not limited to, adenoviral promoters (e.g., the Major Late Promoter (MLP) or promoter within the Major Late transcription unit (MLTU)), adenoviral transcriptional enhancers, adenoviral splice acceptor sites, adenoviral splice enhancers, adenoviral leader sequences (e.g., tripartite leader (TPL) sequences), adenoviral elements that increase RNA stability and/or processing (e.g., cis-acting RNA export elements), and adenoviral poly A signal sequences. The adenoviral transcriptional and/or translational control sequence can be from any adenoviral strain. Thus, an adenoviral vector of the invention can comprise an adenoviral transcriptional and/or translational control sequence derived from a different adenoviral strain. The adenoviral transcriptional and/or translational control sequence can have a wild-type sequence (i.e., a sequence found in a naturally-occurring adenovirus) or variant sequence thereof. Adenoviral transcriptional and/or translational control sequences have been described in the art. For example, adenoviral TPL sequences are described in U.S. Patent Application 2006/0115456; enhancers are described in Massie et al. (1995), Biotechnology 13(6):602; and polyadenylation sequences are discussed in Bhat and Wold (1986), J. Virology 57(3):1155. Additional adenoviral transcriptional and/or translational control sequences can be identified by persons skilled in the art.

In certain embodiments, the transgene sequence is under (i.e., under the control of) an adenoviral MLP. As used herein, “Major Late Promoter (MLP)” is used interchangeably with Major Late transcription unit (MLTU) promoter. In other embodiments, the transgene sequence is under an adenoviral MLP and adenoviral TPL. In other embodiments, the transgene sequence is under an adenoviral MLP and operably linked to an adenoviral splice acceptor sequence. In still other embodiments, the transgene sequence is under an adenoviral MLP and adenoviral TLP, and operably linked to an adenoviral splice acceptor sequence. In certain embodiments, the adenoviral splice acceptor sequence is a non-consensus sequence. Without intending to be limited by theory, it is believed that non-consensus splice acceptors perform better than consensus splice acceptors when they are used in conjunction with the adenoviral MLP. In any of the foregoing embodiments, the transgene sequence can further be operably linked to an adenoviral poly-adenylation signal sequence.

In certain embodiments, the transgene sequence is under (i.e., under the control of) an endogenous adenoviral transcriptional and/or translational control sequence. As used herein, an “endogenous” adenoviral transcriptional and/or translational control sequence is a nucleic acid sequence involved in transcriptional and/or translational regulation that is native to an adenoviral vector and has not been introduced or moved to a new location by means of recombinant technologies.

In certain embodiments, the transgene sequence comprises an exogenous transcriptional and/or translational control sequence. As used herein, an “exogenous” transcriptional and/or translational control sequence refers to either a non-adenoviral transcriptional and/or translational control sequence or an adenoviral transcriptional and/or translational control sequence taken out of its wild-type context and placed into a new context within the heterologous sequence. Examples of exogenous transcriptional and/or translational control sequences include, but are not limited to, promoters functional in mammalian cells (e.g., constitutive promoters, such as a CMV promoter, the Rous sarcoma virus (RSV) LTR promoter, the SV40 promoter, the dihydrofolate reductase (DHFR) promoter, the β-actin promoter, the phosphoglycerol kinase (PGK) promoter, the EF1.alpha. promoter (Invitrogen), etc.), enhancer sequences functional in mammalian cells (e.g., CMV or RSV enhancer sequences), splicing signals, splice enhancers, leader sequences, Kozak sequences, sequences that increase RNA stability and/or processing (e.g., cis-acting RNA export elements, Woodchuck Hepatitis Virus posttranslational regulatory element (WPRE)), poly A signal sequences (e.g., bovine growth hormone (BGH) or SV40 poly A signal sequence), etc. Various suitable transcriptional and/or translational control sequences have been described in the prior art. A suitable CMV promoter has been described, for example, in U.S. Patent Application 2006/0115456. WPRE elements have been described, e.g., in Donello et al. (1998), J. Virology 72(6):5085. WPRE elements must be located within the ORF message, typically between the 3′ end of the gene and the 5′ polyadenylation sequence. Without intending to be limited by theory, it is believed that WPREs function by increasing the efficiency of mRNA translocation from the nucleus, as well as increasing RNA translation and stability. Kozak sequences have also been described, for example, in Kozak, Nucleic Acid Res 15(20), 8125-48 (1987).

Suitable transcriptional and/or translational control sequences, whether adenoviral or otherwise, include naturally-occurring sequences as well as modified forms of such sequences. Such modified forms can include one or more base changes (e.g., deletions, insertions, substitutions) designed to enhance a desirable activity associated with the transcriptional and/or translational control sequence or reduce or eliminate an undesirable activity associated with the endogenous adenoviral transcriptional and/or translational control sequence.

In certain embodiments, the transgene sequence comprises multiple transcriptional or translational control sequences. For example, the transgene sequence can comprise sufficient transcriptional or translational control sequences to ensure expression of an ORF in the transgene sequence upon infection of an appropriate cell (e.g., a human cell) by the adenoviral vector. In certain embodiments, the transgene sequence comprises a promoter (e.g., a CMV promoter) and an adenoviral TPL sequence. In other embodiments, the transgene sequence comprises a promoter (e.g., a CMV promoter), an adenoviral TPL, and an adenoviral poly A signal sequence (e.g., an Ad5 E3A poly A signal sequence). In connection with any of the foregoing embodiments, the transgene sequence can further comprise a Kozak sequence.

In certain embodiments, the transgene sequence comprises one or more transcriptional or translational control sequences for each of two or more ORFs. For example, the transgene sequence can comprise sufficient transcriptional or translational control sequences to ensure expression of each of two or more ORFs. Accordingly, in certain embodiments, the transgene sequence comprises a promoter and a poly A signal sequence for each of two ORFs. The transgene sequence can further comprise an adenoviral TPL and/or a Kozak sequence for each of the ORFs. Alternatively, in certain embodiments, the transgene sequence can comprise sufficient transcriptional or translational control sequences to ensure expression of one ORF (e.g., a promoter and/or enhancer and a poly A signal sequence) while comprising a second ORF that is under the control of or operably linked to endogenous adenoviral transcriptional or translational control sequences.

In certain embodiments, the transgene sequence has been optimized to increase or maximize expression and/or translation of at least one ORF. For example, in certain embodiments, an ORF in the transgene sequence has been codon optimized (e.g., for expression in mammalian cells, such as human cells). In one embodiment, the transgene sequence has been codon optimized and is under the control of a non-adenoviral promoter, such as a CMV promoter. In other embodiments, a Kozak sequence operably linked to an ORF is the transgene sequence has been optimized to create, for example, a consensus Kozak sequence. In still other embodiments, the transgene sequence has been optimized to remove potential inhibitory sequences, such as exonic splice silencers or insulator sequences (e.g., sequences that function to organize chromatin and block the long-range effects of promoters and/or enhancers). Codon optimization and other types of sequence optimization are routine in the art and skilled persons will readily understand how to perform such optimizations.

In some embodiments in which the transgene sequence is under the control of a MLP promoter, the transgene sequence is not codon optimized—i.e., the transgene sequence is the native sequence from the infectious pathogen. For instance, in one embodiment, the adenoviral vector comprises a non-codon optimized transgene sequence under the control of an adenoviral MLP promoter, wherein the adenoviral vector is replication competent and has a partial E3 deletion.

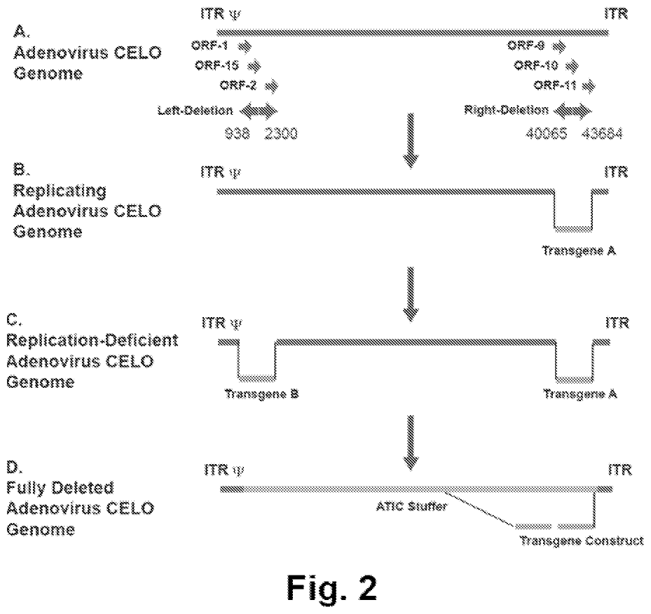

In some embodiments, an AAd-derived replication-deficient gene transfer vector is based on an AAd virus. In one embodiment of an AAd-derived vector a section of the left region of the AAd genome is deleted so that the resulting AAd genome can no longer replicate unless the deleted genome is provided partially or completely in trans by a another genetic construct (FIG. 2C). In another embodiment these deletions of this section of the left region of the AAd genome are replaced in part or in toto by a transgene construct or transgene constructs.

In another embodiment of an AAd-derived replication-deficient gene transfer vector the AAd genome is deleted in the following manner: 1) a section of the right region of the AAd genome is deleted in way that by itself does not prevent the replication of this AAd genome in the absence of any complimentary genetic constructs; and 2) a section of the left region of the AAd genome is deleted so that the resulting AAd genome can no longer replicate unless the deleted genome is provided partially or completely in trans by a another genetic construct (FIG. 2C). In another embodiment these deletions of the AAd genome are replaced in part or in toto by a transgene construct or transgene constructs.

In another embodiment of an AAd-derived replication-deficient gene transfer vector the AAd genome is deleted in a way so that the resulting AAd genome can no longer replicate unless the deleted genome is provided partially or completely in trans by another genetic construct (FIG. 2D). In another embodiment these deletions of the AAd genome are replaced in part or in toto by a transgene construct or transgene constructs. In another embodiment of an AAd-derived replication-deficient gene transfer vector the AAd genome is deleted of all endogenous AAd genes so that only the left and right ITRs together with the packaging signal Ψ remain (FIG. 2D). In these embodiments of a fully deleted “gutted” AAd vector the deleted region is replaced by an inert stuffer sequence in part or in toto. In other embodiments the deleted is replace in part by an inert stuffer sequence and in part by a transgene construct or transgene constructs.

In other embodiments of these AAd-derived replication-deficient gene transfer, the CELO AAd genome is used as basis for the described AAd-derived gene transfer vectors. In other embodiments of these AAd-derived replication-deficient gene transfer, other AAd viruses are used as the basis for the described AAd-deriver gene transfer vectors. They are, but not limited to, the Fowl Aviadenovirus A, the Falcon Aviadenovirus A, the Quail Bronchitis Virus, the Egg Drop Syndrome virus, the hemorrhagic Enteritis virus, the Marble Spleen Disease Virus and the Inclusion Body Hepatitis Virus.

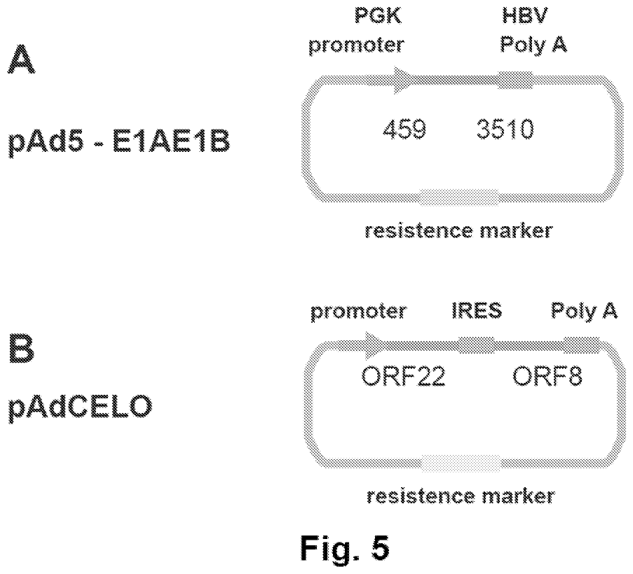

In other embodiments, the complimentary AAd-derived genome fragmented required to mediate the replication and the packaging of a partially deleted AAd-derived vector are composed of the section deleted from the partially deleted repliction-deficient AAd derived vector (FIG. 5). In other embodiments, the complimentary AAd-derived genome fragmented required to mediate the replication and the packaging of a partially deleted AAd-derived vector are composed of some or all sections deleted from the partially deleted repliction-deficient AAd derived vector. In other embodiments, the complimentary AAd-derived genome fragmented required to mediate the replication and the packaging of a partially deleted AAd-derived vector are provided by one or more genetic constructs of some or all sections deleted from the partially deleted repliction-deficient AAd derived vectors.

In other embodiments the complimentary AAd-derived genome fragments are derived from the CELO AAd genome. In other embodiments the complimentary AAd-derived genome fragments are derived from other AAd genomes. They are, but not limited to, the Fowl Aviadenovirus A, the Falcon Aviadenovirus A, the Quail Bronchitis Virus, the Egg Drop Syndrome virus, the hemorrhagic Enteritis virus, the Marble Spleen Disease Virus and the Inclusion Body Hepatitis Virus.

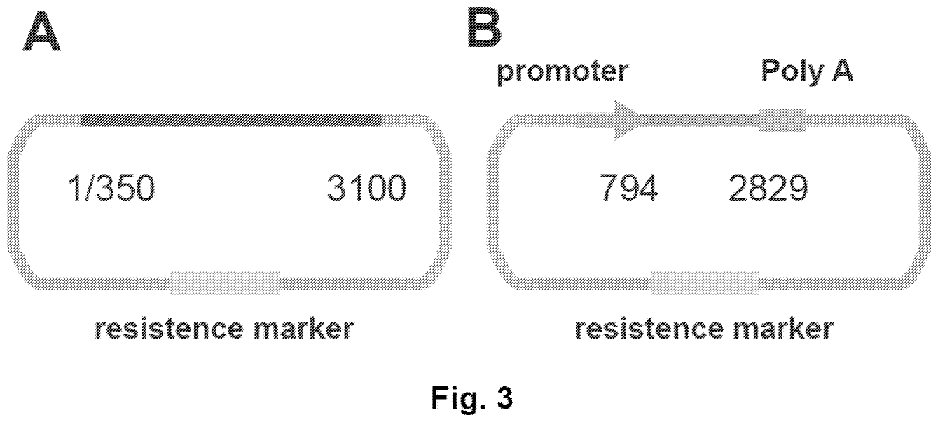

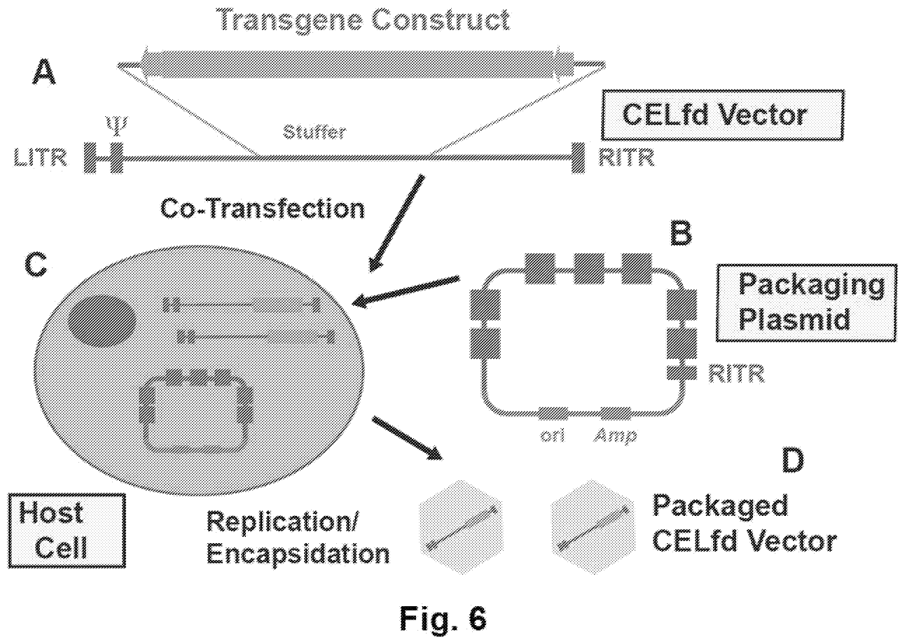

In other embodiments the complimentary AAd-derived genome fragment called packaging construct required to enable the replication of a fully deleted “gutted” AAd-derived vector is composed of an entire or partial AAd-derived genome in all cases deleted of the packaging signal Ψ (FIGS. 3 and 6). In other embodiments the complimentary AAd-derived genome fragment called packaging construct required to enable the replication of a fully deleted “gutted” AAd-derived vector is composed of an entire or partial AAd-derived genome in all cases deleted of one or both ITRs and also the packaging signal Ψ (FIGS. 3 and 6).

In other embodiments, host cells used to replicate and encapsidate AAd-derived gene transfer vectors are eukaryotic cells, such as, but not limited to, human cells. In other embodiments, host cells used to replicate and encapsidate AAd-derived gene transfer vectors are eukaryotic cells derived from animals of the class aves or birds. In other embodiments, host cells used to replicate and encapsidate AAd-derived gene transfer vectors are cells, such as the chemically induced chicken hepartocarcinoma cell line (LMH).

In other embodiments, the complimentary AAd-derived genome fragmented required to mediate the replication and the packaging of a replication-deficient AAd-derived vectors is transiently expressed in host cells. In other embodiments, the complimentary AAd-derived genome fragmented required to mediate the replication and the packaging of a replication-deficient AAd-derived vectors is stably expressed in host cells.

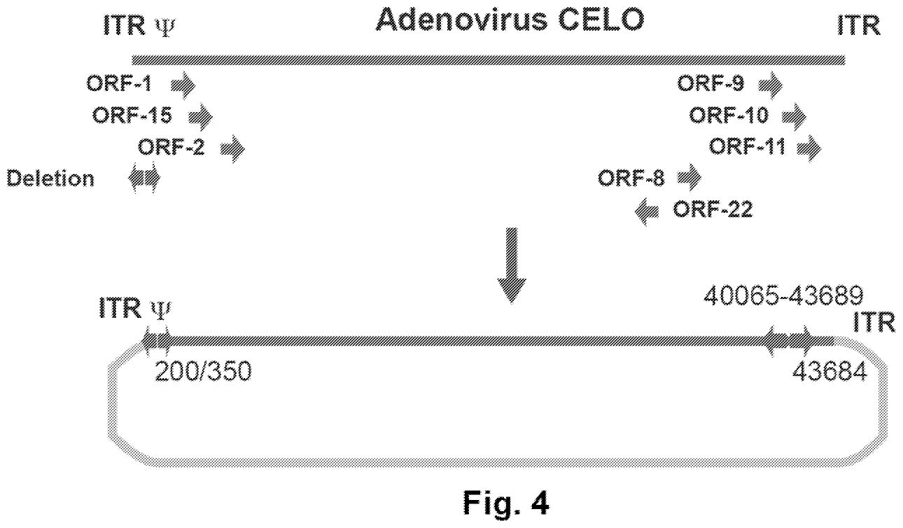

In other embodiments, the host cells used to replicate and encapsidate AAd-derived gene transfer vectors are modified to stably carry an expression cassette carrying genetic material of the left side of the AAd genome (FIG. 4). This expression cassette contains a promoter, such as but not limited to, a PGK promoter and a poly-adenylation site, such as but not limited to, a HSV poly-adenylation site. In other embodiments the AAd genome used to modify host cells is derived from an AAd virus, such as, but not limited to, the CELO virus, the Fowl Aviadenovirus A, the Falcon Aviadenovirus A, the Quail Bronchitis Virus, the Egg Drop Syndrome virus, the hemorrhagic Enteritis virus, the Marble Spleen Disease Virus and the Inclusion Body Hepatitis Virus.

In other embodiments, the host cells used to replicate and encapsidate AAd-derived gene transfer vectors are modified to stably carry an expression cassette carrying genetic material of the left side of a non-AAd genome, such as, but not limited to, an adenovirus from another class of animals.

In other embodiments, a partially deleted AAd-derived replication-deficient vector is produced in the following manner: 1) the AAd-derived genome is released from its cloning vector so that it consists of a linear DNA molecule bordered by the left and right ITRs; 2) the AAd-derived complimentary genetic construct necessary to enable replication of the replication-deficient AAd-derived genome is released from it cloning vector; 3) both genetic constructs are co-transfected into a host cell; 4) the transfected host cell is incubated in a way that the AAd-derived replication-deficient vector is replicated and encapsidated; and 5) the encapsidated AAd vector is released from the cells and harvested.

In another embodiment, the host cell is modified to stably carry and express the AAd-derived complimentary genetic construct necessary to enable replication of the replication-deficient AAd-derived genome. Into this modified host cell, the AAd-derived genome released from its cloning vector is transfected, the transfected host is cell incubated in a way that the AAd-derived replication-deficient vector is replicated and encapsidated; and the encapsidated AAd vector is released from the cells and harvested.

In other embodiments, a fully deleted “gutted” AAd-derived replication-deficient vector is produced in the following manner: 1) the fully deleted “gutted” AAd-derived genome is released from its cloning vector so that it consists of a linear DNA molecule bordered by the left and right ITRs; 2) the complimentary AAd-derived genome fragment called packaging construct required to enable the replication of a fully deleted “gutted” AAd-derived vector is provided in its expression vector; 3) both genetic constructs are co-transfected into host cell modified to stably carry and express the AAd-derived complimentary genetic construct necessary to enable replication of the fully deleted “gutted” AAd-derived genome; 4) the transfected host cell is incubated in a way that the AAd-derived replication-deficient vector is replicated and encapsidated; and 5) the encapsidated AAd vector is released from the cells and harvested.

In some embodiments, an adenoviral vector of the invention comprises a transgene sequence under the control of an adenoviral promoter (e.g., Major Late Promoter), wherein the transgene sequence encodes an antigen from influenza, Bacillus, HIV, HPV, togavirus (e.g. Dengue Fever virus), Shigella, Mycobacterium, Streptococcus, or Plasmodium. In one embodiment, the transgene sequence encodes H1 HA, H3 HA, H5 HA, or B HA antigen from influenza. In another embodiment, the transgene sequence encodes protective antigen or a modified protective antigen from Bacillus anthracis. In another embodiment, the transgene sequence encodes an envelope protein (e.g. gp160, gp140, gp120), modified envelope protein, or a gag protein from HIV. In yet another embodiment, the transgene sequence encodes a L1 protein, L2 protein, E6 protein, E7 protein or fusions thereof from HPV, including HPV16 and HPV18. In still another embodiment, the transgene sequence encodes CSP, Pfs48/45, MSP1, MSP (C-term, p42), or LSA1 from Plasmodium. In some embodiments, the transgene sequence encodes Ag85, ESAT, HspX, or combinations thereof from Mycobacterium. In other embodiments, the transgene sequence encodes PSSP, r56Karp protein, or an invasion protein (e.g., IpaB, IpaC, or IpaD protein) from Shigella. In still further embodiments, the adenoviral vector can further comprise an adenoviral tripartite leader sequence. For instance, the transgene sequence can be under the control of an adenoviral MLP and tripartite leader, wherein the transgene sequence encodes an antigen from influenza, Bacillus, HIV, HPV, togavirus (e.g. Dengue Fever virus), Shigella, Mycobacterium, Streptococcus, or Plasmodium.

In other embodiments, an adenoviral vector of the invention comprises a transgene sequence under the control of a non-adenoviral promoter (e.g., CMV promoter, RSV LTR promoter, SV40 promoter, DHFR promoter, P-actin promoter, PGK promoter, the EF1.alpha. promoter), wherein the transgene sequence encodes an antigen from influenza, Bacillus, HIV, HPV, togavirus (e.g. Dengue Fever virus), Shigella, Mycobacterium, Streptococcus, or Plasmodium. For instance, in one embodiment, the transgene sequence is under the control of a CMV promoter and encodes an antigen from influenza, Bacillus, or HIV. In one particular embodiment, the transgene sequence is codon-optimized sequence from influenza, Bacillus, or HIV. In another embodiment, the transgene sequence is a native sequence from influenza, Bacillus, or HIV. In another embodiment, the transgene sequence encodes H1 HA, H3 HA, H5 HA, B HA, NP, or M1 antigen from influenza. In another embodiment, the transgene sequence encodes protective antigen or a modified protective antigen from Bacillus anthracis. In yet another embodiment, the transgene sequence encodes an envelope protein (e.g. gp160, gp140, gp120), modified envelope protein, or a gag protein from HIV. In some embodiments, the adenoviral vector can further comprise an adenoviral tripartite leader sequence. For instance, the transgene sequence can be under the control of a CMV promoter and adenoviral tripartite leader, wherein the transgene sequence encodes an antigen from influenza, Bacillus, HIV, HPV, togavirus (e.g. Dengue Fever virus), Shigella, Mycobacterium, Streptococcus, or Plasmodium.

In certain embodiments, an adenoviral vector of the invention comprises a second transgene sequence. Thus, in certain embodiments, the adenoviral vector of invention comprises both a transgene sequence and a second transgene sequence. Alternatively, the adenoviral vector of the invention can comprise a second transgene sequence in lieu of the transgene sequence.

The transgene sequence can have a structure as described above for the transgene sequence and can be inserted into the adenoviral genome in any manner described above. Thus, in certain embodiments, the transgene sequence can encode a full-length antigen or a fragment thereof (e.g., a domain, unit(s) of secondary structure, conserved epitope, B-cell, HTL, or CTL epitope, or combinations thereof). In some embodiments, the transgene sequence encodes a therapeutic protein, such as a cytokine or growth factor or other protein that stimulates the immune system. For instance, in one embodiment, the transgene sequence encodes a protein that stimulates white blood cells, such as granulocyte macrophage colony stimulating factor (GM-CSF). In some embodiments, the transgene sequence encodes an antigen from an infectious pathogen and the transgene sequence encodes a therapeutic protein. In one particular embodiment, the transgene sequence encodes an influenza antigen (e.g., H1 HA, H3 HA, H5 HA, or B HA antigen) and the transgene sequence encodes a protein that stimulates white blood cells (e.g., GM-CSF). In certain embodiments, the transgene sequence is inserted into the same region of the adenoviral vector as the transgene sequence (e.g., such that the first and transgene sequences are located proximal to one another). In other embodiments, the first and transgene sequences are inserted into different regions of the adenoviral vector.

The transgene sequence can also be integrated into an adenoviral ORE. In certain embodiments, the adenoviral ORF encodes an adenoviral structural protein (e.g., a capsid protein, such as hexon protein or fiber protein). Thus, in certain embodiments, the transgene sequence is integrated into an adenoviral hexon ORF, wherein the resulting fusion of hexon ORF and heterologous sequences encodes a chimeric hexon protein. In other embodiments, the transgene sequence is integrated into an adenoviral fiber ORF, wherein the resulting fusion of fiber ORF and heterologous sequences encodes a chimeric fiber protein. In general, a chimeric hexon or fiber protein of the invention will retain hexon or fiber function (e.g., form hexon capsomeres or fibers and contribute to capsid formation) while presenting new antigens of the surface of the resulting adenoviruses. The presentation of new antigens of the surface of recombinant adenoviruses of the invention is advantageous because it helps to avoid problems with pre-existing adenovirus immunity in the general population, which can reduce the efficacy of the adenoviral-based vaccines. In addition, the presentation of antigens from infectious pathogens on the surface of the recombinant adenoviruses can broaden the immune response stimulated by the adenoviral-based vaccines of the invention by presenting a greater variety of infectious pathogen antigens to the immune system of a subject taking the vaccine.

Accordingly, in certain embodiments, the transgene sequence is integrated into the ORF of an adenoviral structural protein (e.g., a capsid protein, such as hexon or protein), wherein the transgene sequence encodes an antigen from an infectious pathogen. The infectious pathogen and antigen thereof can be as described above. In certain embodiments, the antigen is from an influenza surface protein, such as M2 (e.g., an external domain, fragment, or epitope of M2). In certain embodiments, the M2 antigen is selected from the set of M2 peptide sequences. In certain embodiments, the transgene sequence encodes more than one of the M2 peptide sequences listed in table 4. For example, the transgene sequence can encode at least two M2 sequences from H1, H2, and/or H3 influenza strains, H5 influenza strains, H7 influenza strains, or H9 influenza strains. Alternatively, the transgene sequence can encode M2 sequences from a plurality of different influenza serotypes. In other embodiments, the transgene sequence can encode one or more copies of an influenza Matrix sequence or influenza NP sequence. In still other embodiments, the influenza antigen is a HTL or CTL epitope. For example, the transgene sequence can encode one or more HTL epitopes.

The amount of sequence that can be inserted into a single hexon HVR depends upon the particular HVR (e.g., HVR1, HVR2, etc.) and the length of the HVR. In general, the insertion can code for a polypeptide sequence corresponding to the length of the HVR polypeptide sequence (if the HVR sequence is being replaced) plus an additional 0 to 75, 1 to 70, 2 to 65, 3 to 60, 4 to 55, or 5 to 50 amino acids. Hexon HVR insertions have been described, e.g., for Ad5 in Matthews et al. (2008), Virology Journal 5:98.

Sequences encoding antigens from infectious pathogens can replace hexon HVRs such that the hexon sequences and antigen sequences are adjacent to one another. As used herein in this context, the term “adjacent” refers to an in-frame fusion between the hexon coding sequences and the antigen coding sequences wherein there is no linker sequence connecting the hexon and antigen sequences. Alternatively, a linker sequence can be used to connect the hexon and antigen sequences. In certain embodiments, the linker sequence is a sequence encoding the tri-peptide “LGS.” The linker sequence can be included, e.g., at the beginning and end of the antigen sequence, as shown in FIG. 12. Without intending to be bound by theory, it is believed that the LGS linker sequences provide structural flexibility, improve the stability of the resulting hexon fusion protein, and/or reduce the immunogenicity of the junctions between the hexon protein sequences and the protein sequences encoded by the heterologous sequence. In other embodiments, the linker sequence encodes the peptide sequence “GAAA” (SEQ ID NO: 352) or “NAA.” Such linker sequences can be used in combination, e.g., with the GAAA sequence on the N-terminal end and the “NAA” sequence on the C-terminal end of the protein encoded by the heterologous sequence. Other appropriate linker sequences can be identified by persons skilled in the art.

In certain embodiments, an adenoviral vector of the invention comprises a third heterologous sequence. Thus, in certain embodiments, the adenoviral vector of invention comprises a first, second, and third heterologous sequence. Alternatively, the adenoviral vector of the invention can comprise a second and a third heterologous sequence. The third heterologous sequence can have a structure as described above for the transgene sequence or the transgene sequence, and can be inserted into the adenoviral genome in any manner described above.

Techniques for constructing, genetically manipulating, and propagating recombinant adenoviral vectors are disclosed in the Examples set forth below. See also, e.g., WO 2008/010864, U.S. Patent Application 2006/0115456, and U.S. Pat. No. 6,127,525, the contents of which are incorporated herein by reference.

In another aspect, the present invention provides vaccines comprising one or more adenoviral vectors of the invention. As used herein, the term “vaccine” refers to a composition that comprises an adenoviral vector of the invention and a carrier. In certain embodiments, the adenoviral vector is a virus. In other embodiments, the adenoviral vector is the genome alone and does not include the adenoviral capsid. In certain embodiments, the carrier is an adjuvant. Examples of such adjuvants include, but are not limited to, salts, such as calcium phosphate, aluminum phosphate, calcium hydroxide and aluminum hydroxide; natural polymers such as algal glucans (e.g., beta glucans), chitosan or crystallized inulin; synthetic polymers such as poly-lactides, poly-glycolides, poly lacitide-co-glycolides or methylacrylate polymers; micelle-forming cationic or non-ionic block copolymers or surfactants such as Pluronics, L121, 122 or 123, Tween 80, or NP-40; fatty acid, lipid or lipid and protein based vesicles such as liposomes, proteoliposomes, ISCOM and cochleate structures; and surfactant stabilized emulsions composed of synthetic or natural oils and aqueous solutions. In certain embodiments, a vaccine of the invention, upon administration to a subject, is capable of stimulating an immune response (e.g., a humoral immune response, cellular immune response, or both) in the subject. In certain embodiments, the immune response includes a measurable response (e.g., a measurable humoral or cellular immune response, or combination thereof) to an epitope encoded by a heterologous sequence inserted or integrated into an adenoviral vector of the vaccine. In certain embodiments, a vaccine of the invention is capable of providing protection against an infectious pathogen or against cancer. For example, in certain embodiments, the vaccine is capable of stimulating an immune response against one or more antigens (e.g., encoded by a heterologous sequence) such that, upon later encountering such an antigen, the subject receiving the vaccine has an immune response that is stronger than it would have been if the vaccine had not been administered previously. In some embodiments, a vaccine of the invention is capable of providing protection against an infectious pathogen or cancer in a subject with pre-existing immunity to adenovirus. In other embodiments, a vaccine of the invention is capable of ameliorating a pathogen infection or cancer and/or reducing at least one symptom of a pathogen infection or cancer. For instance, in one embodiment, the vaccine of the invention induces a therapeutic immune response against one or more antigens encoded by a heterologous sequence such that symptoms and/or complications of a pathogen infection or cancer will be alleviated, reduced, or improved in a subject suffering from such an infection or cancer.

The adenoviral vectors used for the vaccines can be prepared and formulated for administration to a mammal in accordance with techniques well known in the art. Formulations for parenteral, such as, but not limited to, intramuscular, intravenous, subcutaneous and intracutaneous, or enteral, such as, but not limited to, oral administrations have been developed for adenoviral vectors.

Oral administration can consist of capsules or tablets containing a predetermined amount of a recombinant adenoviral vector of the invention; liquid solutions, such as an effective amount of the pharmaceutical dissolved in ingestible diluents, such as water, saline, orange juice, and the like; suspensions in an appropriate liquid; and suitable emulsions. The adenoviral vectors of the invention can, for example, be formulated as enteric coated capsules for oral administration, as previously described, in order to bypass the upper respiratory tract and allow viral replication in the gut. See, e.g., Tacket et al., Vaccine 10:673-676, 1992; Horwitz, in Fields et al., eds., Fields Virology, third edition, vol. 2, pp. 2149-2171, 1996; Takafuji et al., J. Infec. Dis. 140:48-53, 1979; and Top et al., J. Infec. Dis. 124:155-160, 1971. Alternatively, the adenoviral vectors can be formulated in conventional solutions, such as sterile saline, and can incorporate one or more pharmaceutically acceptable carriers or excipients. The pharmaceutical composition can further comprise other active agents.

In certain embodiments, formulations of the invention comprise a buffered solution comprising adenoviral vectors (e.g., viruses) in a pharmaceutically acceptable carrier. A variety of carriers can be used, such as buffered saline, water and the like. Such solutions are generally sterile and free of undesirable matter. These compositions can be sterilized by conventional, well known sterilization techniques, or can be sterile filtered. The compositions may contain pharmaceutically acceptable auxiliary substances as required to approximate physiological conditions such as pH adjusting and buffering agents, tonicity adjusting agents and the like, for example, sodium acetate, sodium chloride, potassium chloride, calcium chloride, sodium lactate and the like.

Pharmaceutically acceptable carriers can contain a physiologically acceptable compound that acts, e.g., to stabilize the composition or to increase or decrease the absorption of the virus and/or pharmaceutical composition. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of any co-administered agents, or excipient, or other stabilizers and/or buffers. Detergents can also be used to stabilize the composition or to increase or decrease absorption. One skilled in the art will appreciate that the choice of a pharmaceutically acceptable carrier, including a physiologically acceptable compound depends, e.g., on the route of administration of the adenoviral preparation and on the particular physio-chemical characteristics of any co-administered agent.

The adenoviral vectors can also be administered in a lipid formulation, more particularly either complexed with liposomes or to lipid/nucleic acid complexes or encapsulated in liposomes. The vectors of the current invention, alone or in combination with other suitable components, can also be made into aerosol formulations to be administered via inhalation. The vaccines can also be formulated for administration via the nasal passages. Formulations suitable for nasal administration, wherein the carrier is a solid, include a coarse powder having a particle size, for example, in the range of about 10 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active ingredient. In some embodiments, the adenoviral vectors of the invention can be formulated as suppositories, for example, for rectal or vaginal administration.

In another aspect, the invention provides methods of inducing an immune response to any infectious pathogen described herein in a subject comprising administering to the subject a vaccine of the invention. In one embodiment, the invention provides a method of vaccinating a subject against an infectious pathogen comprising administering a sufficient amount of a vaccine of the invention to a subject at risk for being infected by an infectious pathogen. In another embodiment, the subject has an infection induced by the infectious pathogen. Thus, for instance, in one embodiment, the present invention provides a method of inducing a therapeutic immune response in a subject experiencing an infection induced by an infectious pathogen. In some embodiments, one or more symptoms or complications of the infection is reduced or alleviated in the subject following administration of the vaccine. The vaccines of the invention can be used to vaccinate human or veterinary subjects.

The vaccines of the invention can be administered alone, or can be co-administered or sequentially administered with other immunological, antigenic, vaccine, or therapeutic compositions. Such compositions can include other agents to potentiate or broaden the immune response, e.g., IL-2 or other cytokines which can be administered at specified intervals of time, or continuously administered (see, e.g., Smith et al., N Engl J Med 1997 Apr. 24; 336(17):1260-1; and Smith, Cancer J Sci Am. 1997 December; 3 Suppl 1:S137-40). The vaccines or vectors can also be administered in conjunction with other vaccines or vectors. For example, an adenovirus of the invention can be administered either before or after administration of an adenovirus of a different serotype. An adenovirus preparation may also be used, for example, for priming in a vaccine regimen using an additional vaccine agent.

The adenoviral formulations can be delivered systemically, regionally, or locally. Regional administration refers to administration into a specific anatomical space, such as intraperitoneal, intrathecal, subdural, or to a specific organ, and the like. Local administration refers to administration of a composition into a limited, or circumscribed, anatomic space such as an intratumor injection into a tumor mass, subcutaneous injections, intramuscular injections, and the like. One of skill appreciates that local administration or regional administration can also result in entry of the viral preparation into the circulatory system. Typical delivery routes include parenteral administration, e.g., intradermal, intramuscular or subcutaneous routes. Other routes include oral administration, including administration to the oral mucosa (e.g., tonsils), intranasal, sublingual, intravesical (e.g., within the bladder), rectal, and intravaginal routes. For delivery of adenovirus, administration can often be performed via inhalation. Aerosol formulations can, for example, be placed into pressurized, pharmaceutically acceptable propellants, such as dichlorodifluoro-methane, nitrogen and the like. They can also be formulated as pharmaceuticals for non-pressurized preparations such as in a nebulizer or an atomizer. Typically, such administration is in an aqueous pharmacologically acceptable buffer as described above. Delivery to the lung can also be accomplished, for example, using a bronchoscope.

The vaccines of the invention can be administered in a variety of unit dosage forms, depending upon the intended use, e.g., prophylactic vaccine or therapeutic regimen, and the route of administration. With regard to therapeutic use, the particular condition or disease and the general medical condition of each patient will influence the dosing regimen.

The amount and concentration of virus and the formulation of a given dose, or a “therapeutically effective” dose can be determined by the veterinarian or clinician. A therapeutically effective dose of a vaccine is an amount of adenovirus that will stimulate an immune response to the protein(s) encoded by the heterologous nucleic acid included in the viral vector. The dosage schedule, i.e., the dosing regimen, will depend upon a variety of factors, e.g., the general state of the patient's health, physical status, age and the like. The state of the art allows the clinician to determine the dosage regimen for each individual patient. Adenoviruses have been safely used for many years for human vaccines. See, e.g., Franklin et al., supra; Jag-Ahmade et al., J. Virol., 57:267, 1986; Ballay et al., EMBO J. 4:3861, 1985; PCT publication WO 94/17832. These illustrative examples can also be used as guidance to determine the dosage regimen when practicing the methods of the invention.

Single or multiple administrations of adenoviral formulations can be administered as prophylactic or therapeutic vaccines. In one embodiment, multiple doses (e.g., two or more, three or more, four or more, or five or more doses) are administered to a subject to induce or boost a protective or therapeutic immune response. The two or more doses can be separated by periodic intervals, for instance, one week, two week, three week, one month, two month, three month, or six month intervals.

In yet another aspect, the invention also provides kits that contain the vectors, vector systems or vaccines of the invention. The kits can, for example, also contain cells for growing the adenoviruses of the invention. The kits can also include instructional material teaching methodologies for generating adenoviruses using the kits and, for vaccines, can include instruction for indication of dosages, routes and methods of administration and the like.

The following examples illustrate various aspects of the present invention. The examples should, of course, be understood to be merely illustrative of only certain embodiments of the invention and not to constitute limitations upon the scope of the invention which is defined by the claims that are appended at the end of this description.

Having described the disclosure in detail, it will be apparent that modifications and variations are possible without departing from the scope of the claims.

### EXAMPLES

The following non-limiting examples are provided to further illustrate the present disclosure.

### Example 1

Construction of Recombinant Replication-Deficient CELO AAd-Derived Gene Transfer Vectors.

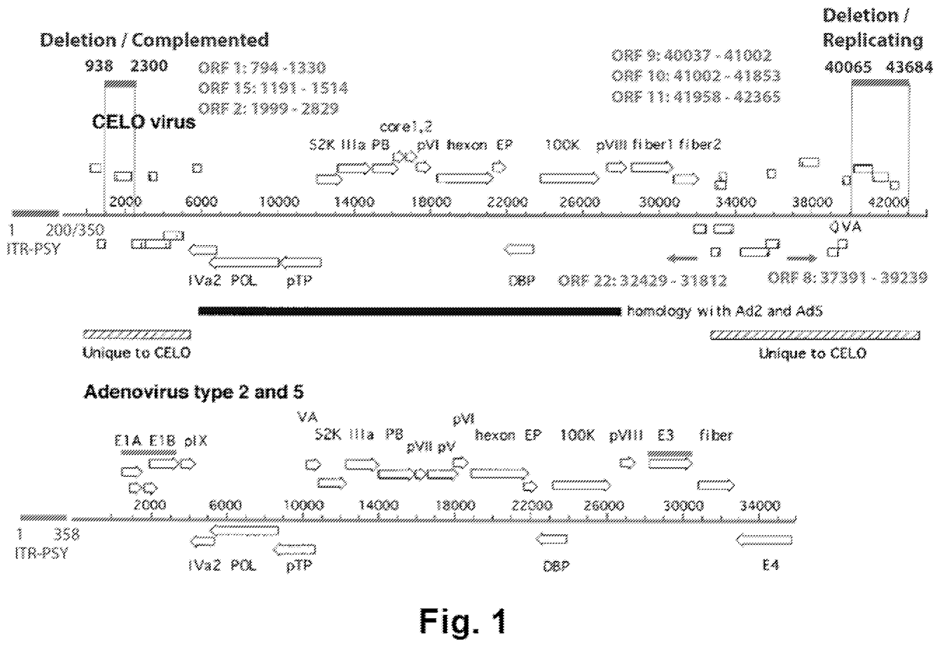

The genome of the aviadenovirus CELO is a linear DNA of approximately 44 kb of length (FIG. 1). It is bordered by inverted terminal repeats (ITR) of approximately 120 bp in lengths. Upstream from the left ITR a packaging signal Ψ is located approximately within the nucleotides 70 through 200.

Others have demonstrated that nucleotides 400065 through 43684 of the CELO genome can be deleted or replaced a transgene construct without destroying the ability of the CELO genome to replicate and to be packaged in host cells (FIG. 2A). This upstream genome region loosely corresponds to the open reading frames 9, 10 and 11.

Deletion or replacement of the CELO genome between the nucleotides 938 and 2300 abolishes the ability of the CELO genome to replicate by itself (FIG. 2B). This downstream genome region loosely corresponds to the open reading frames 1, 15 and 2. Replication and encapsidation of this partially deleted CELO genome can be enabled by the presence of a complimentary CELO genome fragment composed of a genome fragment encompassing the open reading frames 1, 15 and 2 with a promoter sequence upstream to the open reading frame 1. For instance such aa complimentary CELO genome fragment may be consist of, but not limited two, a CELO genome fragment encompassing nucleotides 1 through 3100 or 250 though 3100.

As depicted in a diagrammatic manner in FIG. 2C, a replication-deficient CELO based aviadenoviral vector, named CELrd, can be constructed by the deletion of a genome fragment on the downstream side of the CELO genome that functionally and/or partially deletes the open reading frames 1, 15 and 2 or that loosely corresponds to a genome fragment composed of the nucleotides 794 through 2829. Transgene constructs can be integrated in this deletion that either carry their own promoter and poly-adenylation sites or use the respective sites found in this region of the CELO genome.

As depicted in a diagrammatic fashion in FIG. 2C, another replication-deficient CELO-based aviadenoviral vector can be constructed that carries a second deletion to the down-stream deletion described above. The CELO genome can also be deleted of a genome fragment on the upstream side of the CELO genome that functionally and/or partially deletes the open reading frames 9, 10 and 11 or that loosely corresponds to a genome fragment composed of the nucleotides 40037 through 42365. Transgene constructs can be integrated in this deletion that either carry their own promoter and poly-adenylation sites or use the respective sites found in this region of the CELO genome. Restriction enzyme sites will be placed outside the vector construct adjacent to the ITRs so that the vector genome can be released by restriction enzyme cuts.

### Example 2

Construction of Fully Deleted “Gutted” Replication-Deficient CELO AAd-Derived Gene Transfer Vectors.

As depicted in a diagrammatic manner in FIG. 2D, a CELO AAd-derived gene transfer vector can be constructed by deleting large fragments of the CELO genome and replacing them by a non-adenoviral stuffer sequence. The deleted fragments can be replaced my transgene constructs that are composed of the transgenes of interest linked to promoter and poly-adenylation sites or that use promoter and poly-adenylation sites found within the CELO genome. More than one transgene construct can be integrated into the deleted CELO genome. The CELO genome can be deleted of all CELO genes leaving the ITRs, the packaging signal Ψ and non-coding CELO sequences in place. The deleted sequences are replaced by an inert stuffer and/or transgene expression construct. Such CELO vectors are denoted fully deleted and/or “gutted” and are called CELfd. The remaining CELO sequences loosely correspond to the CELO genome of the nucleotides 1 through 200, or 1 through 350 together with a deletion of nucleotides 43604 through 43804. Restriction enzyme sites will be placed outside the vector construct adjacent to the ITRs so that the vector genome can be released by restriction enzyme cuts.

### Example 3

Construction of a complimentary genetic construct enabling the replication and encapsidation of replication-deficient CELO AAd-derived vectors.

To enable the replication and encapsidation of partially deleted CELO AAd-derived vectors of the CELrd-type described in Example 1, a segment of the CELO genome loosely complementary to the down-stream deletion described for a CELrd-type vector has to be present in the packaging cell or host cells upon introduction of the CELrd genome. This complimentary CELO genome segment has to provide the genetic information and thus the protein encoded by the open reading frames 1, 15 and 2. This complimentary CELO genome segment has to at least encompass the CELO genome of nucleotides 794 through 2829 in a form that provides for the expression of protein encoded by the open reading frames 1, 15 and 2. Expression of these proteins may be enabled by a CELO genome fragment, such as, but not limited to a CELO genome fragment of nucleotides 1 through 3100, or 250 through 3100 (FIG. 3A). Expression of these proteins may be enabled by an expression vector that uses a heterologous promoter and heterologous poly-adenylation site, or heterologous promoters and heterologous poly-adenylation sites, to facilitate the expression of the open reading frames 1, 15 and 2 (FIG. 3B).

### Example 4

Construction of a Packaging Expression Vector Enabling the Replication and Encapsidation of a Fully Deleted “Gutted” CELO AAd-Derived Gene Transfer Vector Genome

To enable the replication and encapsidation of CELO AAd-derived gene transfer vectors that carry large deletion of the CELO genome or are fully deleted “gutted” CELO vectors, such as CELfd vector, described in Example 2, a packaging expression vector has to be provided to the packaging cell or host cell together with the CELO vector genome.

This packaging expression vector is deleted of the packaging signal Ψ which is found in the CELO genome region around nucleotides 70 through 200 or 350 (FIG. 4). The packaging expression plasmid can also be deleted of one or both of the inverted terminal repeats and segments of the CELO genome corresponding to a partial or complete deletion of reading frames 8, 10 and 11 (FIG. 4).

### Example 5

Construction of CELO genome fragments to enhance the function of packaging or host cells.

The activity of packaging cells or host cells will be enhanced by an expression construct to expresses CELO gene, such as gene encoded by the open reading frames 22 and 8 (pAdCELO). Corresponding to an expression vector used to enable packaging of human adenoviral vectors that carries the genes for the adenoviral E1A and E1B, this expression construct will be design to express the genes of the reading frames 22 and 8 (FIG. 5A). An example of such an expression vector is given in FIG. 5B. It will carry a promoter, either a heterologous or an adenoviral one, the open reading frames 22 and 8, possibly linked by internal ribosomal entry site, followed by poly-adenylation site, either a heterologous or an adenoviral one. This expression construct may either be stable integrated into the genome of the packaging or host cells or co-transfected into the packaging or host cell during replication and encapsidation of a CELO AAd-derived vector.

### Example 6

Replication and encapsidation of replication-deficient CELO AAd-derived gene transfer vectors (FIG. 6).

As exemplified here for a fully deleted “gutted” CELO AAd-derived gene transfer vector, the genome of the CELO AAd-derived vector, here a CELfd genome, will be released from its cloning vector by a restriction enzyme cut (FIG. 6A). Together with the packaging expression construct (FIG. 6B), it will be co-transfected into packaging or host cells (FIG. 6C), such as LMH cells, that may have been modified by carrying a pAdCELO expression construct. After an incubation of a few days, the encapsidated CELfd vectors are released.

A partially deleted CELO AAd-derived gene transfer vector of the type CELrd will be produced in the following way. Its genome will be released by a restriction enzyme cut. Together with the complimentary genetic construct as described in Example 3, it will be co-transfected into packaging or host cells, such as LMH cells, that may have been modified by carrying a pAdCELO expression construct. After an incubation of a few days, the encapsidated CELfd vectors are released. It may be possible to stably integrate the complimentary genetic construct of Example 3 into the packaging or host cells. Then the CELrd can be replicated and packaged by transfection of the packaging or host cells with the CELrd genome or by the transduction of the packaging or host cells with encapsidated CELrd vectors.

### Example 7

Engineering of replication-deficient CELO AAd-vectored vaccine against avian influenza.

A CELrd of CELfd constructs is loaded with a transgene construct composed of a cytomegalovirus promoter followed by a hemagglutinin gene, an internal ribosomal entry site, a neuraminidase gene and a poly-adenylation site wherein the hemagglutinin and the neuraminidase genes are derived from an influenza virus of the H5N1 or H7N9 serotype. Birds and also animals of other species can be vaccinated with this construct delivered as an intramuscular, intravenous, subcutenous, intranasal or enteral vaccine. In the case of birds the vaccine may be given by injecting the fertilized egg.

Alternatively a CELfd construct is loaded with more than one transgene expression construct of the design above so that vaccination against influenza of different serotypes can be effected with a single construct.

Alternatively a CELfd construct is loaded with transgene derived from different infectious diseases to be used as a combination vaccine.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

In view of the above, it will be seen that the several objects of the disclosure are achieved and other advantageous results attained.

As various changes could be made in the above products and methods without departing from the scope of the disclosure, it is intended that all matter contained in the above description shall be interpreted as illustrative and not in a limiting sense.

What is claimed is:

1. An aviadenoviral gene transfer vector comprising a deleted avidenoviral genome wherein the aviadenoviral vector is replication deficient and has a partial deletion of its genome.

2. The aviadenoviral gene transfer vector of claim 1, wherein the aviadenoviral vector is derived from one of the different species and serotypes identified as aviadenoviruses.

3. The avidenoviral gene transfer vector of claim 1, wherein the aviadenoviral vector is derived from the Fowl Aviadenovirus A, the Falcon Aviadenovirus A, the Quail Bronchitis Virus, the Egg Drop Syndrome virus, the hemorrhagic Enteritis virus, the Marble Spleen Disease Virus and the Inclusion Body Hepatitis Virus.

4. The aviadenoviral gene transfer vector of claim 3, wherein the avidenoviral genome is functionally deleted of open reading frames corresponding to the E1A and E1B regions.

5. The aviadenoviral gene transfer vector of claim 1, wherein the aviadenoviral vector is derived the chicken the embryo lethal orphan (CELO) virus.

6. The avidenoviral gene transfer vector of claim 5, wherein the avidenoviral genome partially deleted of the open reading frames 1, 15 and 2.

7. The avidenoviral gene transfer vector of claim 5, wherein the open reading frames 1, 15 and 2 are partially replaced by heterologous transgenes.

8. An aviadenoviral complimentary genome construct, wherein the open reading frames 1, 15 and 2 of the CELO genome are expressed.

9. An aviadenoviral complimentary genome construct of claim 9, wherein the open reading frames 1, 15 and 2 are carried with a CELO genome fragment.

10. An aviadenoviral complimentary genome construct of claim 9, wherein the open reading frames 1, 15 and 2 are expressed from a expression vector.

11. A packaging and host cell for aviadenoviral vectors comprising an avian cell.

12. A packaging and host cell of claim 11, wherein the cell has been transfected with a construct that expressed genes corresponding to the adenoviral E1A and E1B regions.

13. A packaging and host cell of claim 11, wherein the cell has been transfected with a construct that encompassed the CELO open reading frames 22 and 8.

14. A replication and encapsidation scheme for a replication-deficient aviadenoviral vector comprising:
(1) a replication-deficient aviadenoviral vector;
(2) a complimentary aviadenoviral construct;
(3) an avian packaging or host cell; and
(4) co-transfection of a replication-deficient aviadenoviral vector and a complimentary aviadenoviral construct into the avian packaging or host cell.

15. An aviadenoviral packaging expression vector, comprising a CELO aviadenovirus derived genome deleted of the packaging signal Ψ.

16. An aviadenoviral packing expression vector of claim 15, wherein its genome is deleted of at least one of its ITRs.

17. An aviadenoviral packaging expression vector of claim 15, wherein its genome is functionally deleted of the open reading frames 9, 10 and 11.

18. A replication and encapsidation scheme for fully deleted “gutted” aviadenoviral vector comprising:
(1) a fully deleted “gutted” aviadenoviral vector;
(2) an aviadenoviral packaging expression vector construct;
(3) an avian packaging or host cell; and
(4) co-transfection of a fully deleted “gutted” aviadenoviral vector and an aviadenoviral packaging expression vector construct into the avian packaging or host cell.

19. An encapsidated replication-deficient aviadenoviral vector of claim 14, wherein the vector is used as a gene transfer vector.

20. An encapsidated replication-deficient aviadenoviral vector of claim 14, wherein the vector is used for vaccination.

21. An encapsidated replication-deficient aviadenoviral vector of claim 18, wherein the vector is used as a gene transfer vector.

22. An encapsidated replication-deficient aviadenoviral vector of claim 18, wherein the vector is used for vaccination.
